(19) United States
(12) Patent Application Publication
(10) Pub. No.: US 2010/0035858 A1
(43) Pub. Date: Feb. 11, 2010

(54) Novel Compositions and Methods for Binding and Inhibiting 5-HT4 Receptor

(75) Inventors: Stephen Roth, Gladwyne, PA (US); David Zopf, Wayne, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Immune Control, Inc.

(21) Appl. No.: 12/504,533

(22) Filed: Jul. 16, 2009

Related U.S. Application Data
(60) Provisional application No. 61/135,273, filed on Jul. 18, 2008.
(60) Provisional application No. 61/143,553, filed on Jan. 9, 2009.

(51) Int. Cl. A61K 31/5415; C07D 417/06; A61K 31/397; A61P 25/00

(52) U.S. Cl. 514/210.21; 544/45; 514/225.8

(57) ABSTRACT

The present invention relates to compositions that bind to 5-HT4 receptors and inhibit proliferation of activated lymphocytes. The invention also provides methods for inhibiting proliferation and inducing cell death in activated immune cells, as well methods for treating diseases associated with activated immune cells by administering 5-HT4 receptor ligands.

### CROSS-REFERENCE TO RELATED APPLICATIONS

This application claims priority under 35 U.S.C. §119(e) to U.S. Provisional Application No. 61/135,273, filed on Jul. 18, 2008 and U.S. Provisional Application No. 61/143,553, filed on Jan. 9, 2009, which applications are incorporated by reference herein in its entirety.

### BACKGROUND OF THE INVENTION

Serotonin (also referred to as 5-hydroxytryptamine or 5-HT) is a neurotransmitter that has been strongly implicated in the pathophysiology and treatment of a wide variety of neuropsychiatric disorders. Serotonin exerts its effects through a diverse family of serotonin receptor molecules (referred to herein as “5-HT receptors” or “5-HTRs”). Classically, members of the serotonin receptor family have been grouped into seven (7) subtypes pharmacologically, i.e., according to their specificity of various serotonin antagonists. Thus, while all the 5-HT receptors specifically bind with serotonin, they are pharmacologically distinct and are encoded by separate genes. To date, fourteen (14) mammalian serotonin receptors have been identified and sequenced. More particularly, these fourteen separate 5-HT receptors have been grouped into seven (7) pharmacological subtypes, designated 5-HT1, 5-HT2, 5-HT3, 5-HT4, 5-HT5, 5-HT6, and 5-HT7. Several of the subtypes are further subdivided such that the receptors are grouped pharmacologically as follows: 5-HT1A, 5-HT1B, 5-HT1D, 5-HT1E, 5-HT1F, 5-HT2A, 5-HT2B, 5-HT2C, 5-HT3A, 5-HT3B, 5-HT4, 5-HT5, 5-HT6, 5-HT7. The nucleic and amino acid sequences of the receptors do not predict their pharmacological groupings.

Of the fourteen different mammalian serotonin receptors that have been cloned, all but one are members of the G-protein coupled receptor superfamily. Serotonin receptors 5-HT1A, 5-HT1B, and 5-HT1D inhibit adenylate cyclase, and 5-HT2 receptors activate phospholipase C pathways, stimulating breakdown of polyphosphoinositides. The 5-HT2 receptor belongs to the family of rhodopsin-like signal transducers that are distinguished by a seven-transmembrane configuration and functional linkage to G-proteins. The 5-HT3 receptor family includes ligand-gated ion channel receptors that have four putative transmemebrane domains (TMDs).

Serotonin regulates a wide variety of sensory, motor and behavioral functions in the mammalian CNS, including behaviors such as learning and memory, sleep, thermoregulation, motor activity, pain, sexual and aggressive behaviors, appetite, neuroendocrine regulation, and biological rhythms. Serotonin has also been linked to pathophysiological conditions such as anxiety, depression, obsessive-compulsive disorders, schizophrenia, suicide, autism, migraine, emesis, alcoholism and neurodegenerative disorders. This biogenic amine neurotransmitter is synthesized by neurons of the brain stem that project throughout the CNS, with highest density in basal ganglia and limbic structures (Steinbusch, 1984, In: Handbook of Chemical Neuroanatomy 3:68-125, Bjorklund et al., Eds., Elsevier Science Publishers, B. V.).

Studies have suggested that serotonin may play a role in the immune system since data demonstrate that serotonin receptors are present on various cells of the immune system. There have been reports in the literature about the immunomodulatory effects of adding serotonin exogenously to mitogenically stimulated lymphocyte cultures. Under some circumstances, serotonin has been shown to activate T cells (Foon et al., 1976, J. Immunol. 117:1545-1552; Kut et al., 1992, Immunopharmacol. Immunotoxicol. 14:783-796; Young et al., 1993, Immunology 80:395-400), whereas other laboratories report that high concentrations of added serotonin inhibit proliferation (Slauson et al., 1984, Cell. Immunol. 84:240-252; Khan et al., 1986, Int. Arch. Allergy Appl. Immunol. 81:378-380; Mossner & Lesch, 1998, Brain, Behavior, and Immunity 12:249-271).

Of the fourteen known pharmacologically distinct serotonin receptors, lymphocytes express type 2A, type 2B, type 2C, type 6 and type 7 on resting cells (Ameisen et al., 1989, J. Immunol. 142:3171-3179; Stefulj et al., 2000, Brain, Behavior, and Immunity 14:219-224), and type 1A and type 3 receptors are up-regulated upon activation (Aune et al., 1993, J. Immunol. 151:1175-1183; Meyniel et al., 1997, Immunol. Lett. 55:151-160; Stefulj et al., 2000, Brain, Behavior, and Immunity 14:219-224).

The involvement of the 5-HT1A receptors in human and mouse T cells has also been demonstrated (Aune et al., 1990, J. Immunol. 145:1826-1831; Aune et al., 1993, J. Immunol. 151:1175-1183; Aune et al., 1994, J. Immunol. 153:1826-1831). These studies established that IL-2-stimulated human T cell proliferation could be inhibited by a blockade of tryptophan hydroxylase, i.e., the first enzyme involved in the conversion of tryptophan to serotonin, and that the inhibition could be reversed by the addition of 5-hydroxytryptophan. Furthermore, human T cell proliferation was blocked in vitro with a 5-HT1A-specific receptor antagonist. In a murine model, a type 1A receptor antagonist, but not a type 2 receptor antagonist, was able to inhibit the in vivo contact sensitivity response, but not antibody responses, to oxazalone.

PCT Publication No. WO 2002/078643 discloses a method of inhibiting the interaction of serotonin with serotonin type 2 receptors on a cell using a specific antagonist, and/or inhibiting the signal(s) transduced through the serotonin type 2 receptor to inhibit activation of T cells. PCT Publication No. WO 2003/106660 discloses the use of fluphenazine, an antagonist of 5-HT(1B/1D) and 5-HT(2C) receptors, for inhibiting proliferation and inducing cell death in lymphocytes. PCT Publication No. WO 2008/027521 discloses the use of various fluphenazine derivatives for inhibiting proliferation and inducing cell death in activated lymphocytes. PCT Publication No. WO 2006/138038 discloses methods of treating psoriasis in a human where the method comprises the intralesional administration of fluphenazine, or a derivative thereof, to a psoriatic lesion on the skin of the patient.

5-HT4 receptor is a G protein coupled receptor (GPCR) having seven putative transmemebrane domains. Many medically significant biological processes are mediated by signal transduction pathways that involve G-proteins. The family of G-protein coupled receptors (GPCRs) includes receptors for hormones, neurotransmitters, growth factors, and viruses. Specific examples of GPCRs include receptors for such diverse agents as dopamine, calcitonin, adrenergic hormones, endothelin, cAMP, adenosine, acetylcholine, serotonin, histamine, thrombin, kinin, follicle stimulating hormone, opsins, endothelial differentiation gene-1, rhodopsins, odorants, cytomegalovirus, G-proteins themselves, effector proteins such as phospholipase C, adenyl cyclase, and phosphodiesterase, and actuator proteins such as protein kinase A and protein kinase C. GPCRs possess seven conserved membrane-spanning domains connecting at least eight divergent hydrophilic loops. GPCRs, also known as seven transmembrane receptors, have been characterized as including these seven conserved hydrophobic stretches of about 20 to 30 amino acids, connecting at least eight divergent hydrophilic loops. Most GPCRs have single conserved cysteine residues in each of the first two extracellular loops, which form disulfide bonds that are believed to stabilize functional protein structure. The seven transmembrane domains are designated as TMD1, TMD2, TMD3, TMD4, TMD5, TMD6, and TMD7. TMD3 is being implicated with signal transduction. Phosphorylation and lipidation (palmitoylation or farnesylation) of cysteine residues can influence signal transduction of some GPCRs. Most GPCRs contain potential phosphorylation sites within the third cytoplasmic loop and/or the carboxy terminus. For several GPCRs, such as the beta-adrenergic receptor, phosphorylation by protein kinase A and/or specific receptor kinases mediates receptor desensitization.

The 5-HT4 receptor was first characterized by Dumuis et al. (1988, Molec. Pharm. 34: 880-887) in mouse colliculi neurons. Subsequently, Eglen et al. (1995, Trends Pharm. Sci. 16: 391-398) showed that 5-HT4 receptor mediates widespread effects in central and peripheral nervous systems. Blondel et al. (1997, FEBS Lett. 412: 465-474) used PCR, based on primers to the central region of rat 5-HT4 receptor, to clone a human 5-HT4 receptor.

The 5-HT4 receptor is widely distributed in the body, in the periphery as well as in the central nervous system. In the periphery it is found in the gastrointestinal tract, for example in the esophagus (Moummi et al., 1992 Eur J. Pharmacol. 1992 216:47-52), the ileum (Buchheit and Buhl, 1991 Eur J. Pharmacol. 205:203-208) and colon (Elswood et al., 1991Br J Pharmacol. 102:811-816). It is also present in the atrium (Kaumann. et al., Br J Pharmacol. 100:879-885), the bladder (Candura et al., 1996 Br J Pharmacol. 118:1965-1970) and the adrenal glands. In the rat brain, 5-HT4 receptor mRNA has been discovered by in situ hybridization in the olfactory tubercle, the striatum and the hippocampus (Vilaro et al., 1996 Brain Res Mol Brain Res 43:356-360). The wide distribution in different tissues of the 5-HT4 receptor is paralleled by a wide variety of 5-HT4 variants caused by alternative splicing of exons.

The h5-HT4(a) receptor was cloned from human heart (Blondel et al., 1997 FEBS Lett. 412:465-474; Claeysen et al., 1997 Neuroreport. 8:3189-3196). The h5-HT4(b) receptor was cloned from a library (Van de Wyngaert et al., 1997 J. Neurochem. 69:1810-1819). These two receptors, h5-HT4(a) and h5-HT4(b), when transiently expressed in COS-7 cells, exhibited a conventional 5-HT4 receptor pharmacological profile. Other splice variants include h5-HT4(c) and h5-HT4(d) receptors. The isoform h5-HT4(c) receptor presents a high number of putative phosphorylation sites (one for protein kinase C, one for protein kinase A/protein kinase G, and two for casein kinase II), all contained in the last 25 residues of the amino acid sequence (U.S. Pat. No. 6,506,580). 5-HT4(e) receptor has been isolated from human heart (Mialet et al., Br J Pharmacol. 129:771-781).

There exists a long-felt need to develop novel compounds and therapies for treating diseases related to activated immune cell and immune cell proliferation, for example, diseases related to activated T cells, B cells, natural killer (NK) cells, dendritic cells (DC), macrophages, monocytes, neutrophils, a T cell, a B cell, a natural killer cell, a dendritic cell, and a macrophage, a monocyte, a neutrophil, a eosinophil, and/or a basophil. In addition, there is a long-felt need to develop novel compounds without the side effects related to other serotonin receptor ligands. The present invention meets these needs.

### BRIEF SUMMARY OF THE INVENTION

The present invention provides a composition comprising an effective amount of a 5-HT4 receptor ligand, wherein the ligand has a Ki value of less than about 1 μM for binding thereof to a 5-HT4 receptor, and further wherein the ligand is capable of modulating activity of an immune cell.

In one embodiment, the ligand inhibits proliferation of an immune cell when the ligand binds to a 5-HT4 receptor on the immune cell.

In one embodiment, the immune cell is selected from the group consisting of a T cell, a B cell, a natural killer cell, a dendritic cell, and a macrophage, a monocyte, a neutrophil, a eosinophil, and a basophile.

In another embodiment, the ligand does not substantially modulate cells of the central nervous system.

In another embodiment, the ligand does not substantially cross the blood-brain barrier.

The present invention also provides a method of treating a disease characterized by abnormal immune cell proliferation. The method comprising administering to a mammal in need thereof a composition comprising an effective amount of a 5-HT4 receptor ligand, wherein the ligand has a Ki value of less than about 1 μM for binding thereof to a 5-HT4 receptor, and further wherein the ligand is capable of modulating activity of an immune cell. Preferably, mammal is a human.

In one embodiment, the immune cell is selected from the group consisting of a T cell, a B cell, a natural killer cell, a dendritic cell, a macrophage, a monocyte, a neutrophil, a eosinophil, and a basophile.

In another embodiment, the administered composition does not substantially modulate central nervous system function of the mammal.

In yet another embodiment, the ligand does not substantially cross the blood-brain barrier of the mammal.

The invention also provides a method of treating rheumatoid arthritis in a mammal. The method comprises administering to a mammal in need thereof an effective amount of a composition comprising a 5-HT4 receptor ligand, wherein the ligand has a Ki value of less than about 1 μM for binding thereof to said 5-HT4 receptor, and further wherein the ligand is capable of modulating activity of an immune cell.

In one embodiment, the ligand inhibits proliferation of an immune cell associated rheumatoid arthritis when the ligand binds to a 5-HT4 receptor on the immune cell. The immune cell is selected from the group consisting of a T cell, a B cell, a natural killer cell, a dendritic cell, and a macrophage, a monocyte, a neutrophil, a eosinophil, and a basophile.

In one embodiment, the administered composition does not substantially modulate central nervous system function of the mammal.

In another embodiment, the ligand does not substantially cross the blood-brain barrier of the mammal. Preferably, the mammal is a human.

The present invention also provides a method of treating rheumatoid arthritis in a mammal. The method comprises administering to a mammal in need thereof an effective amount of a composition comprising a 5-HT4 receptor ligand in combination with a therapeutic agent. Preferably, the therapeutic agent is a rheumatoid arthritis medicament and the ligand has a Ki value of less than about 1 μM for binding thereof to a 5-HT4 receptor. More preferably, the ligand is capable of modulating activity of an immune cell.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a chart depicting exemplary results from experiments of screening candidate compounds for their ability to inhibit proliferation of RPMI cells. ICI-847 and ICI-1260 depict exemplary proliferation curves associated with active compounds capable of inhibiting proliferation of RPMI cells. ICI-953 depicts an exemplary proliferation curve associated with an inactive compound with respect to inhibiting proliferation of RPMI cells.

FIG. 2 is a schematic of a rat collagen-induced arthritis model used to test the efficacy of candidate compounds. The schematic depicted is adapted from Rosloniec et al., 2001 Curr. Protoc. Immunol. Chapter 15: Unit 15.5.

FIG. 3, comprising FIGS. 3A and 3B, is a series of charts summarizing the efficacy results from screening the ability of candidate compounds to reduce ankle diameter as an indication for treating arthritis. Dosing was at 10 mg/kg ip.

FIG. 4 is a chart depicting the seven (7) most active compounds tested in the collagen-induced arthritis animal model for reducing ankle diameter.

FIG. 5, comprising FIGS. 5A through 5G, is a series of charts depicting dose-response curves with respect to reducing ankle diameter in an animal arthritis model. FIG. 5A is a dose-response curve for ICI-1260; FIG. 5B is a dose-response curve for ICI-1259; FIG. 5C is a dose-response curve for ICI-848; FIG. 5D is a dose-response curve for ICI-847; FIG. 5E is a dose-response curve for ICI-849; FIG. 5F is a dose-response curve for ICI-715; FIG. 5G is a dose-response curve for ICI-761.

FIG. 6 is a chart depicting dose-response curves for the seven (7) compounds that exhibited greater efficacy than the control compound, Enbrel, for reducing the ankle diameter in the animal arthritis model.

FIG. 7, comprising FIGS. 7A through 7D, is a series of charts depicting the selective binding of candidate compounds to 5-HT receptors. Each candidate compound was tested for selective binding to 5-HT1B, 2A, 2B, 2C, 3, 4, 6, and 7 receptors at concentrations of 10 μM and 0.1 μM.

FIG. 8 is a chart demonstrating the selective binding of the tested compounds (ICI-848, 1259, and 1260) versus fluphenazine (ICI-1175; control) to different types of 5-HT receptors. The tested compounds exhibited a decreased binding to 5-HT1A and 5-HT2A receptors as compared to the ability of fluphenazine to bind 5-HT1A and 5-HT2A receptors. In contrast, the tested compounds exhibited an increased binding to 5-HT4 receptor as compared to the ability of fluphenazine to bind5-HT4 receptor.

FIG. 9, comprising FIGS. 9A through 9D, is a series of charts depicting percent reduction in ankle diameter versus 5-HT receptor binding. Percent reduction in ankle diameter was observed to correlate with the ability of the test compound to bind 5-HT4 receptor only. It was observed that compounds that reduced ankle diameter in the rheumatoid arthritis rat model had a greater binding affinity to 5-HT4 receptor than 5-HT5A, 5-HT6, and 5-HT7 receptors.

FIG. 10, comprising FIGS. 10A through 10D is a series of charts depicting that the ability for a test compound to bind 5-HT4 receptor correlates with the ability for the compound to reduce the ankle diameter in an arthritis animal model occurs at a higher dose (e.g., 10 mg/kg) rather than lower doses (e.g., 2 mg/kg).

FIG. 11, comprising FIGS. 11A through 11C, is a series of charts depicting the Ki values for the tested compounds to bind to 5-HT2A, 5-HT2B, 5-HT2C, 5-HT4, 5-HT6, and 5-HT7 receptors.

FIG. 12 is a chart depicting the ability of the tested compound to bind non-5-HT receptors.

### DETAILED DESCRIPTION OF THE INVENTION

The present invention relates to compositions and methods for inducing cell death in activated immune cells, including, but not limited to, T cells, B cells, NK cells, dendritic cells, macrophages, monocytes, neutrophils, eosinophils, basophils, and the like. In addition, the present invention relates to compositions and methods for inhibiting the proliferation of activated immune cells. As demonstrated by the data disclosed herein, the serotonin receptor ligands disclosed herein reduce symptoms of arthritis in an animal model. Thus, the present invention encompasses methods, compositions and kits for treating various diseases associated with the proliferation and/or activation of immune cells, including, but not limited to lymphomas, myelomas, autoimmune diseases, transplant rejection, inflammatory disease, and the like.

### DEFINITIONS

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

By T cell “activation,” as the term is used herein, is meant that the T cell, when contacted with a compound, molecule, or cell capable of generating an immune response (e.g., a mitogen or antigen), detectably upregulates surface markers, such as CD25, i.e., the IL-2 receptor, initiates a phosphorylation cascade involving p561ck, causes the release of cytokines and interleukins, increases DNA synthesis which can be assessed by, among other methods, assessing the level of incorporation of ³H-thymidine into nascent DNA strands, and causes the cells to proliferate.

Similarly, “activation of a serotonin” receptor, as used herein, means that binding of serotonin with a serotonin receptor on a cell induces the typical cascade of intra and extracellular events associated with such binding.

As used herein, to “alleviate” a disease means reducing the severity of one or more symptoms of the disease.

By the term “allogeneic graft,” as used herein, is meant grafting of any tissue within a species wherein there is a mismatch of an immunological marker, such as, but not limited to, the major histocompatibility complex (MHC), and/or a minor antigen.

The term “allogeneic graft response”, as used herein, means any immune response directed against non-self tissue grafted into a recipient. Grafting procedures include, but are not limited to, administering non-self cells, tissue, or organs during, e.g., bone marrow transplantation, organ transplant, and the like.

The term “apoptosis,” as used herein, means an active process, involving the activation of a preexisting cellular pathway, induced by an extracellular or intracellular signal, causing the death of the cell. In particular, the cell death involves nuclear fragmentation, chromatin condensation, and the like, in a cell with an intact membrane.

By the term “applicator,” as the term is used herein, is meant any device including, but not limited to, a hypodermic syringe, a pipette, an iontophoresis device, a patch, and the like, for administering the ligand of the invention to a mammal.

Inhibition of serotonin signaling is “deleterious” to a cell, as the term is used herein, where the inhibition mediates a detectable decrease in the viability of the cell. Cell viability can be assessed using standard methods that are well-known in the art, including, but not limited to, assessing the level of biomolecular synthesis (e.g., protein synthesis, nucleic acid synthesis, and the like), trypan blue exclusion, MTT reduction, uptake of propidium iodide, exposure of phosphatidylserine on the cell surface, DNA fragmentation and/or ladder formation, and the like.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

By the term “does not substantially cross the blood-brain barrier”, as used herein, means that the inhibitor does not detectably cross the blood-brain barrier as assessed using standard assays such as those disclosed herein, known in the art, or such assays as are developed in the future to determine the permeability of a compound across the blood-brain barrier. Such assays include, but are not limited to, assessing the neuro-psychotropic effects of the compound when administered to an animal. Further, the assays encompass, among other things, assessing the concentration of the compound beyond the barrier, or an art-recognized model of the blood-brain barrier, over time to determine the permeability of the compound through the barrier.

It would be understood by the artisan that an inhibitor can be ab initio impermeable and not cross the blood-brain barrier at a detectable level. Further, it would be understood that an inhibitor of interest can be modified, using techniques well-known in the art, such that it does not detectably cross the blood-brain barrier, or crosses it at a detectably lower level that it did before it was modified. In both instances, whether it loses its ability to cross the blood-brain barrier at a detectable level or loses the ability to cross it at a lower level than before it was modified, the compound is considered to “not substantially cross the blood-brain barrier” for purposes of this section.

By the term “effective amount”, as used herein, is meant an amount of a ligand that is sufficient to bind 5-HT4 receptor or a 5-HT4 like receptor and mediate a detectable change in transmission of signaling via receptor on a cell. Transmission of a signal can be assessed using standard methods well-known in the art, such as, but not limited to, those described elsewhere herein, including, for example, assessing the level of binding of serotonin with a receptor and/or assessing the level of activation of a cell. In some instances, an effective amount of a compound of the invention is an amount sufficient to bind to a target 5-HT receptor or a group of 5-HT receptors to decrease proliferation of an immune cell.

The skilled artisan would understand that the amount varies and can be readily determined based on a number of factors such as the disease or condition being treated, the age and health and physical condition of the mammal being treated, the severity of the disease, the particular compound being administered, and the like. Generally, the dosage will be set between 1 mg/kg and 25 mg/kg. In one embodiment, the drug is administered through intravenous bolus injection. This type of bolus administration can be used to ensure that all of the immunologically relevant cells encounter sufficient quantity of the drug in order to block their receptor-mediated signals. However, the invention is not limited to this method of administration.

By the term “immune reaction,” as used herein, is meant the detectable result of stimulating and/or activating an immune cell.

“Immune response,” as the term is used herein, means a process that results in the activation and/or invocation of an effector function in either the T cells, B cells, natural killer (NK) cells, and/or antigen-presenting cells (APCs). Thus, an immune response, as would be understood by the skilled artisan, includes, but is not limited to, any detectable antigen-specific or allogeneic activation of a helper T cell or cytotoxic T cell response, production of antibodies, T cell-mediated activation of allergic reactions, and the like.

“Immune cell,” as the term is used herein, means any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

By the term “an inhibitor of the interaction of serotonin with a serotonin type 4 receptor,” as used herein, is meant any compound or molecule that detectably inhibits signaling via a serotonin type 4 receptor. Such compounds include a serotonin receptor ligand, an inverse agonist, and the like.

“Instructional material,” as that term is used herein, includes a publication, a recording, a diagram, or any other medium of expression which can be used to communicate the usefulness of the nucleic acid, peptide, and/or compound of the invention in the kit for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

By the term “modulating” an immune response, as used herein, is meant mediating a detectable increase or decrease in the level of an immune response in a mammal compared with the level of an immune response in the mammal in the absence of a treatment or compound, and/or compared with the level of an immune response in an otherwise identical but untreated mammal. The term encompasses perturbing and/or affecting a native signal or response thereby mediating a beneficial therapeutic response in a mammal, preferably, a human.

By the term “modulating” central nervous system function, as used herein, is meant mediating a detectable increase or decrease in the function of the central nervous system in a mammal compared with the level of central nervous system function in the mammal in the absence of a treatment or compound, and/or compared with the level of central nervous function in an otherwise identical but untreated mammal. The term encompasses perturbing and/or affecting a native signal or response thereby mediating a beneficial therapeutic response in a mammal, preferably, a human. In some instances, modulating central nervous system function is associated with modulating the activity of cells of the central nervous system. An example of a cell of the central nervous system is a neuron.

A “receptor” is a compound that binds with a ligand.

A “serotonin antagonist” is a composition of matter which, when administered to a mammal such as a human, detectably inhibits a biological activity attributable to the level or presence of serotonin.

A “serotonin receptor antagonist” is a composition of matter which, when administered to a mammal such as a human, detectably inhibits a biological activity attributable to serotonin binding to a serotonin receptor.

By the term “selective ligand,” as used herein, is meant a chemical agent that has a greater affinity for the target serotonin receptor type than for any other serotonin receptor family member. In some instances, a selective ligand has a greater affinity for a group of target serotonin receptor types. In some instances, the affinity is at least 5-fold more preferential affinity for a target serotonin receptor or a group of target serotonin receptor than other serotonin receptor family members.

A “serotonin receptor” includes a polypeptide that preferably binds with serotonin.

“Serotonin signal,” as the term is used herein, means a change in the balance of any intracellular biochemical pathway as a result of a receptor-mediated interaction with serotonin, a specific drug interaction with any serotonin-specific receptor, or both, that results in the change.

By the term “specifically binds,” as used herein, is meant a receptor that recognizes and binds serotonin family molecules present in a sample (i.e., dopaminergic proteins, adrenergic protein, histamines, melatonin, and serotonin), but does not substantially recognize or bind other molecules in the sample.

By the term “preferably binds,” as used herein, is meant to refer to a compound of the present invention having a greater affinity to a target serotonin receptor type than for any other serotonin receptor family member. In some instances, a compound of the invention can have a greater affinity for a group of target serotonin receptor types. In some instances, the affinity is at least 5-fold more preferential affinity for a target serotonin receptor or a group of target serotonin receptor than other serotonin receptor family members.

To “treat” a disease as the term is used herein, means to reduce the frequency of the disease or disorder reducing the frequency with which a symptom of the one or more symptoms disease or disorder is experienced by an animal.

### DESCRIPTION

The present invention relates to methods, compositions and kits for treating diseases and conditions associated with activated immune cells and the diseases resulting from the activation of immune cells. The present invention encompasses methods for inhibiting and killing activated immune cells, compositions that inhibit and/or kill activated immune cells, compositions that inhibit the proliferation of activated immune cells, and kits for using the methods and compositions of the invention.

The compositions of the present invention include compounds that bind to 5-HT receptor, where the compounds have the chemical formula disclosed elsewhere herein.

In a preferred aspect, the compounds of the invention are able to preferentially bind to 5-HT4 receptor. In some instances, the compounds are able to preferentially bind to both 5-HT4 and 5-HT2B receptors. As demonstrated by the data disclosed herein, the compositions of the present invention are useful in treating an exemplary animal model of an inflammatory disease such as rheumatoid arthritis. Thus, the compounds of the present invention are useful in inhibiting proliferation of immune cells, and are therefore useful in the treatment of diseases where activated and/or proliferating immune cells cause pathology. Examples of diseases associated with activated and/or proliferating immune cells include diseases associated with activated and/or proliferating lymphocytes, including, but not limited to, lymphomas, myelomas, autoimmune diseases, inflammatory diseases, and transplant rejection.

The compositions of the present invention include 5-HT receptor ligands having the chemical formula disclosed elsewhere herein. In some instances, the ligand preferably binds 5-HT4 receptor. In other instances, the ligand preferably binds to both 5-HT4 and 5-HT2B receptors. In yet other instances, the ligand preferably binds 5-HT4, 5-HT2B, and 5-HT6 receptors. In still yet other instances, the ligand preferably binds 5-HT4, 5-HT2B, 5-HT6, and 5-HT7 receptors. The compositions of the invention can further comprise combinations of the 5-HT receptor ligands. In addition, the compositions can further comprise other types of compounds that are capable of inhibiting and/or killing activated immune cells.

One of skill in the art would also appreciate, based upon the disclosure provided herein, that the invention encompasses 5-HT receptor ligands that do not have a substantial effect on central nervous system function. This is because the desired ligands of the invention do not substantially cross the blood-brain barrier. One skilled in the art would understand that because serotonin receptors are found on neural cells and on cells of the immune system, it is desirable, but not necessary, to inhibit signaling via serotonin receptor or serotonin-like receptor on an immune cell while not affecting serotonin signaling via a serotonin receptor on a neural cell.

As demonstrated by the data disclosed herein, the compositions of the present invention inhibit and/or kill activated immune cells by, among other things, inducing cell death in activated immune cells. In addition, the ligands of the present invention inhibit proliferation of lymphocytes, such as T cells and B cells, and are therefore useful in the treatment of diseases where activated and/or proliferating lymphocytes cause pathology. Such diseases include, but are not limited to, lymphomas, myelomas, autoimmune diseases, inflammatory diseases, and transplant rejection.

The methods of the present invention encompass methods of inhibiting and/or killing an activated immune cell, and methods of inhibiting the proliferation of an immune cell. This is because, as demonstrated by the data disclosed herein, the methods of the invention cause a dose and time dependent inhibition of proliferating immune cells, as well as dose and time dependent cell death in immune cells. The methods of the present invention further comprise methods of treating a patient suffering from a disease associated with an activated immune cells, such as rheumatoid arthritis. Such diseases are known in the art and are disclosed elsewhere herein. The methods of the invention are based, in part, on the novel finding that 5-HT receptor ligands, such as those disclosed herein, are useful in inhibiting and/or killing activated immune cells. Preferably, the 5-HT receptor ligand binds at least 5-HT4 receptor. In other preferred embodiments, the 5-HT receptor ligand binds at least 5-HT4 and 5-HT2B receptors. In another embodiment, the ligand binds at least 5-HT4, 5-HT2B, and 5-HT6 receptors. In yet other embodiments, the ligand binds at least 5-HT4, 5-HT2B, 5-HT6, and 5-HT7 receptors. This is because the invention is partly related to the discovery that binding to the 5-HT4 receptor correlates with a therapeutic effect such as diminishing the immunological characteristics of the disease model.

However, the invention should not be limited to ligands that bind 5-HT4 receptors. This is because 5-HT4 receptor is a seven transmembrane G protein coupled receptor (GPCR) and therefore the ligands of the present invention are capable of binding to other GPCRs, including, but not limited to, receptors for hormones, neurotransmitters, growth factors, dopamine, calcitonin, adrenergic hormones, endothelin, cAMP, adenosine, acetylcholine, serotonin, histamine, thrombin, kinin, follicle stimulating hormone, opsins, endothelial differentiation gene-1, rhodopsins, odorants, cytomegalovirus, phospholipase C, adenyl cyclase, phosphodiesterase, protein kinase A, protein kinase C, and the like.

### I. Compositions

The present invention comprises compositions for inhibiting and/or killing activated immune cells, for inhibiting proliferation in immune cells and for treating diseases associated with such immune cells. One embodiment of the present invention, includes compositions which, as demonstrated by the data disclosed herein, induce cell death in various activated lymphocytes, including T cells and B cells. The compositions of the present invention include a composition of Formula I as well as the compositions disclosed below.

The present invention comprises a compound according to formula I as disclosed herein. As demonstrated by the data disclosed herein, 5-HT receptor ligands having the structure of formula I are useful in the present invention for inhibiting the proliferation of immune cells, and for inducing cell death in immune cells. Also demonstrated by the data disclosed herein, 5-HT receptor ligands having the structure of formula I are useful in the present invention for inhibiting the proliferation of lymphocytes, such as T cells and B cells, and for inducing cell death in lymphocytes. Thus, the compounds of the present invention are useful for treating, among other things, lymphomas, myelomas, autoimmune diseases, inflammatory diseases, transplant rejection, and the like.

A. Formula I:

The present invention comprises the use of an effective amount of a compound according to formula I, including, but not limited to ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, ICI-1505, or a pharmaceutically acceptable salt thereof. Compounds of formula I have the following chemical structure:

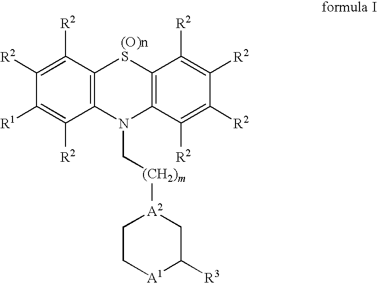

or a pharmaceutically acceptable salt, prodrug or solvate thereof, wherein:

R¹ is independently selected at each occurrence from hydrogen, halogen, (C₁-C₆)alkyl; (C₁-C₆)alkenyl; (C₁-C₆)alkoxy; OH; NO₂; C—N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is CH₂, N((CH₂)ₚNR⁷₂)₂, or NR⁴;

A² is CH or N;

provided that if A¹ is CH₂, then A² is N, and if A² is CH, then A¹ is NR⁴ or N((CH₂)ₚNR⁷₂)₂;

R⁴ is H; (C₁-C₆)alkyl; heteroaryl; (CH₂)ₚOR⁷; (CH₂)ₚNR⁷₂; (CH₂)ₚNHC(O)R⁵; (CH₂)ₚO(CH₂)ₚOR⁷; (CH₂)ₚO(CH₂)ₚNR⁷₂; (CH₂)ₚNR⁴(CH₂)ₚNR⁷₂; (CH₂)ₚO(CH₂)ₚNHC(O)R⁵; (CH₂)ₚNR⁷(CH₂)ₚNHC(O)R⁵; (CH₂)_{q}C(═O)OR⁷₂; (CH₂)_{q}C(═O)NR⁷₂; (CH₂)ₚO(CH₂)_{q}C(═O)OR⁷; (CH₂)ₚO(CH₂)_{q}C(═O)NR⁷₂; (CH₂)ₚNR⁷(CH₂)_{q}C(═O)OR⁷; (CH₂)ₚNR⁷(CH₂)_{q}C(═O)NR⁷₂; (CH₂)ₚR⁸; C(═O)(CH₂)ₚR⁸; (CH₂)ₚO(CH₂)ₚNR⁸, (CH₂)ₚNR⁴(CH₂)ₚNR⁸; (CH₂)_{q}C(═O)NR⁸, (CH₂)ₚO(CH₂)_{q}C(═O)NR⁸, (CH₂)ₚNR⁷(CH₂)_{q}C(═O)NR⁸ or C(═O)(CH₂)ₚNR⁷₂;

R⁵ is (C₁-C₆)alkyl; NR⁷C(═O)(C₁-C₆)alkyl; NR⁷C(═O)O(C₁-C₆)alkyl; NR⁷C(═O)NR⁷₂; CH(R⁶)NR⁷₂; CH(R⁶)NR⁷C(═O)(C₁-C₆)alkyl; (1H-pyrrolidin-2-yl), or CH(R⁶)NR⁷C(═O)O(C₁-C₆)alkyl.

R⁶ is H, (C₁-C₆)alkyl; (C₁-C₆)alkylene-OR⁷; (C₁-C₆)alkylene-NH—C(═NH)—NH₂; (C₁-C₆)alkylene-NR⁷₂; (C₁-C₆)alkylene-SR⁷; benzyl; 4′-hydroxybenzyl; (CH₂)_{q}C(═O)OR⁷; or (CH₂)_{q}C(═O)NR⁷₂;

R⁷ is independently selected at each occurrence from the group consisting of hydrogen and (C₁-C₆)alkyl;

R⁸ is

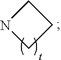

m is independently at each occurrence 1, 2, or 3;

n is 0, 1, or 2;

p is independently at each occurrence 2 or 3;

q is independently at each occurrence 1 or 2; and

t is 1, 2 or 3.

In the definitions of each of the compounds of formula I above:

The term “alkyl”, by itself or as part of another substituent means, unless otherwise stated, a straight, branched or cyclic chain hydrocarbon having the number of carbon atoms designated (i.e. C₁-C₆ means one to six carbons) and includes straight, branched chain or cyclic groups. Examples include: methyl, ethyl, propyl, isopropyl, butyl, isobutyl, tertbutyl, pentyl, neopentyl, hexyl, cyclohexyl and cyclopropylmethyl. Most preferred is (C₁-C₃)alkyl, particularly ethyl, methyl and isopropyl.

The term “alkenyl” employed alone or in combination with other terms, means, unless otherwise stated, a stable monounsaturated or di-unsaturated straight chain, branched chain or cyclic hydrocarbon group having the stated number of carbon atoms. Examples include vinyl, propenyl (allyl), crotyl, isopentenyl, butadienyl, 1,3-pentadienyl, 1,4-pentadienyl, cyclopentenyl, cyclopentadienyl and the higher homologs and isomers. A functional group representing an alkene is exemplified by CH═CHCH₂.

The term “alkylene”, by itself or as part of another substituent means, unless otherwise stated, a divalent straight, branched or cyclic chain hydrocarbon.

The term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, particularly ethoxy and methoxy.

The term “aryl”, employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl; anthracyl; and naphthyl. Preferred are phenyl and naphthyl, most preferred is phenyl.

The term “heteroaryl” refers to a heterocycle having aromatic character. A polycyclic heteroaryl may include one or more rings that are partially saturated. Examples include tetrahydroquinoline and 2,3 dihydrobenzofuryl. For compounds of formula I, the attachment point is understood to be on an atom that is part of an aromatic monocyclic ring or a ring component of a polycyclic aromatic that is itself an aromatic ring.

Examples of heteroaryl groups include: pyridyl, pyrazinyl, pyrimidinyl, particularly 2 and 4 pyrimidinyl, pyridazinyl, thienyl, furyl, pyrrolyl, particularly 2 pyrrolyl, imidazolyl, thiazolyl, oxazolyl, pyrazolyl, particularly 3 and 5 pyrazolyl, isothiazolyl, 1,2,3 triazolyl, 1,2,4 triazolyl, 1,3,4 triazolyl, tetrazolyl, 1,2,3 thiadiazolyl, 1,2,3 oxadiazolyl, 1,3,4 thiadiazolyl and 1,3,4 oxadiazolyl.

Examples of polycyclic heterocycles include: indolyl, particularly 3,4,5 6 and 7 indolyl, indolinyl, quinolyl, tetrahydroquinolyl, isoquinolyl, particularly 1 and 5 isoquinolyl, 1,2,3,4 tetrahydroisoquinolyl, cinnolinyl, quinoxalinyl, particularly 2 and 5 quinoxalinyl, quinazolinyl, phthalazinyl, 1,8 naphthyridinyl, 1,4 benzodioxanyl, coumarin, dihydrocoumarin, benzofuryl, particularly 3,4,1,5 naphthyridinyl, 5,6 and 7 benzofuryl, 2,3 dihydrobenzofuryl, 1,2 benzisoxazolyl, benzothienyl, particularly 3,4, 5,6, and 7 benzothienyl, benzoxazolyl, benzthiazolyl, particularly 2 benzothiazolyl and 5 benzothiazolyl, purinyl, benzimidazolyl, particularly 2 benzimidazolyl, benztriazolyl, thioxanthinyl, carbazolyl, carbolinyl, acridinyl, pyrrolizidinyl, and quinolizidinyl.

The aforementioned listing of heteroaryl moieties is intended to be representative and not limiting.

The term halogen means, unless otherwise stated, a fluorine, chlorine, bromine, or iodine atom, preferably, fluorine, chlorine, or bromine, more preferably, fluorine or chlorine.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —CF₃.

The compounds of formula I or those disclosed herein can be prepared by a person skilled in the art of synthetic organic chemistry. The person skilled in the art knows how to select and implement appropriate synthetic routes. Suitable synthetic methods may be identified by reference to the literature describing synthesis of analogous compounds, and then performing the synthesis of the desired compound following the route used for the analogous compounds, modifying the starting materials, reagents, and reaction conditions as appropriate to synthesizing any particular desired compounds. In addition, reference may be made to sources such as Comprehensive Organic Synthesis, Ed. B. M. Trost and I. Fleming (Pergamon Press 1991), Comprehensive Organic Functional Group Transformations, Ed. A. R. Katritzky, O. Meth Cohn, and C. W. Rees (Pergamon Press, 1996), Comprehensive Organic Functional Group Transformations II, Ed. A. R. Katritzky and R. J. K. Taylor (Editor) (Elsevier, 2nd Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees, and E. F. V. Scriven (Pergamon Press, 1996), the entire disclosures of which are incorporated herein by reference.

It will be understood that when compounds of formula I or those disclosed herein contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention that are efficacious in the treatment of diseases associated with activated and/or proliferating lymphocytes, including, but not limited to, lymphomas, myelomas, autoimmune diseases, and transplant rejection.

The isomers resulting from the presence of a chiral center comprise a pair of non superimposable isomers that are called “enantiomers.” Single enantiomers of a pure compound are optically active, i.e., they are capable of rotating the plane of plane polarized light.

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

By “isolated optical isomer” means a compound that has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well known chiral separation techniques. According to one such method, a racemic mixture of a compound having the structure of Formula I or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

The present invention further comprises compositions for inhibiting and/or killing activated immune cells, for inhibiting proliferating immune cells, and for treating diseases associated with such immune cells. One embodiment of the present invention includes compositions which, as demonstrated by the data disclosed herein, induce cell death in a variety of activated immune cells, including T cells and B cells. The compositions of the present invention include the compositions disclosed below.

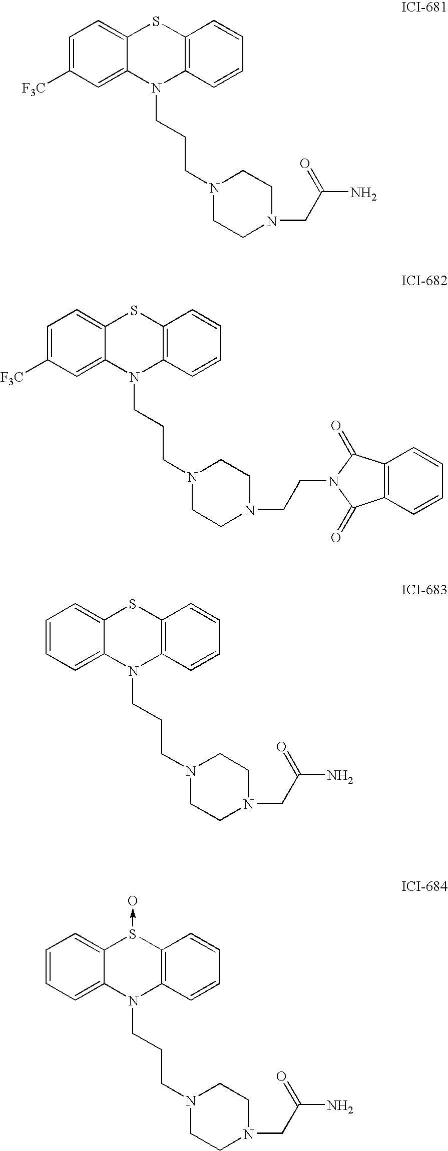

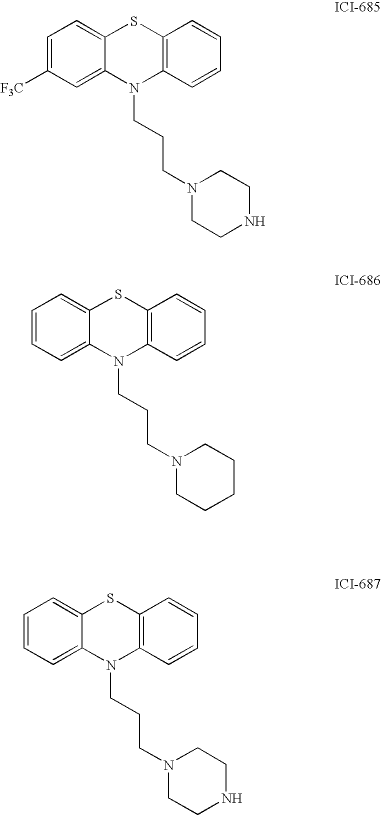

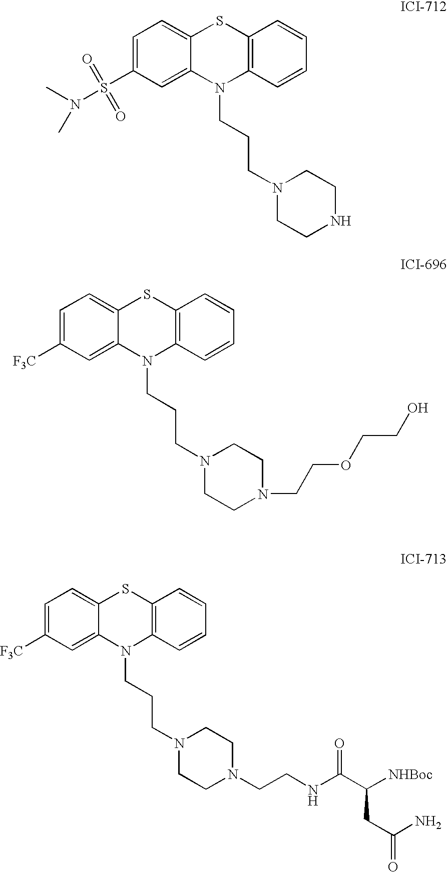

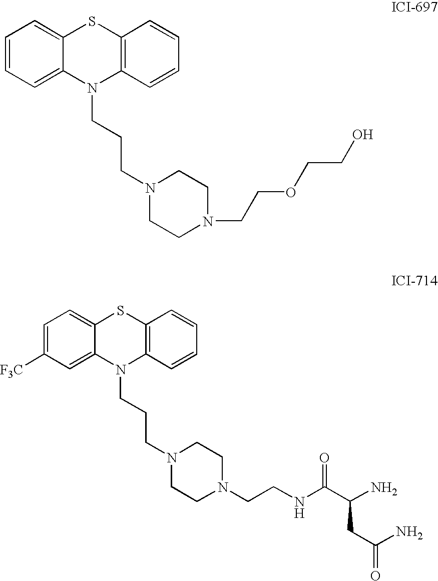

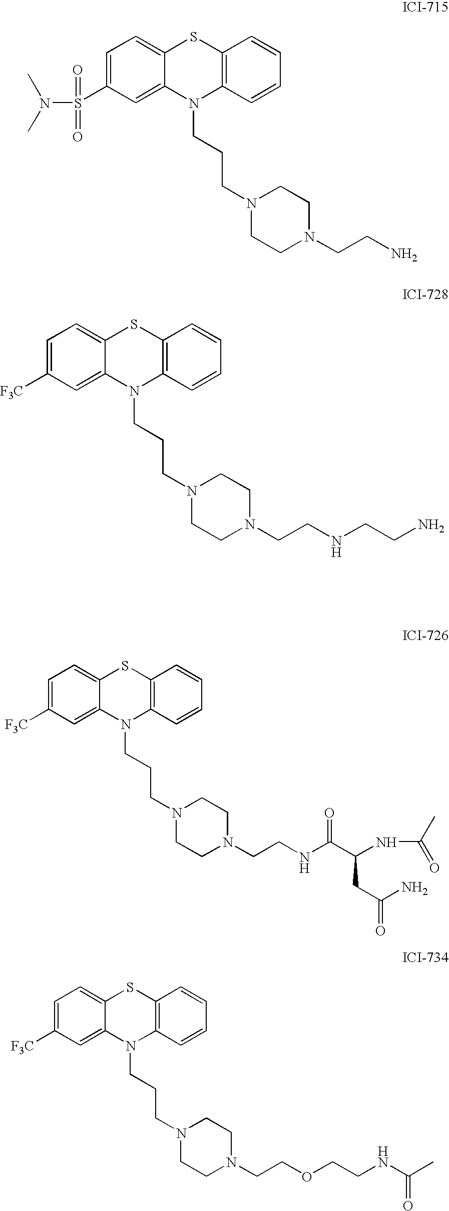

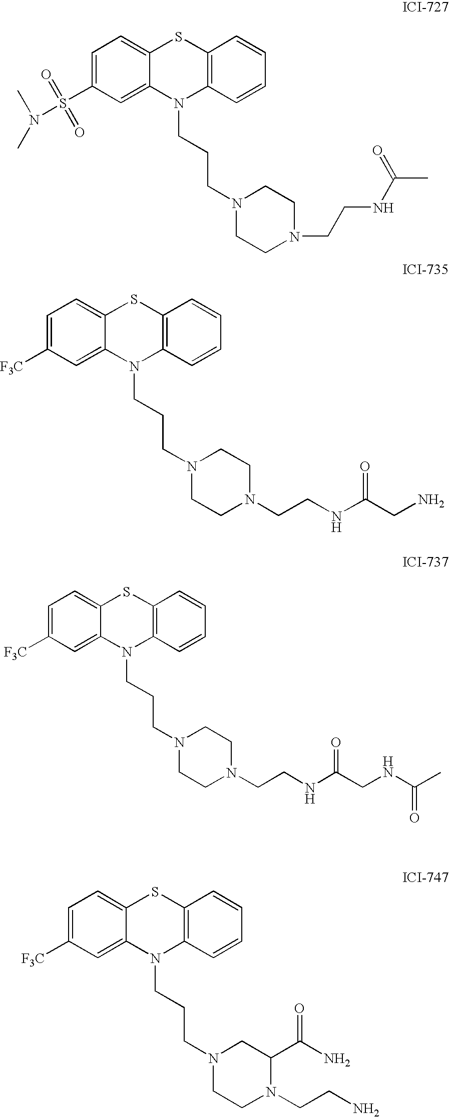

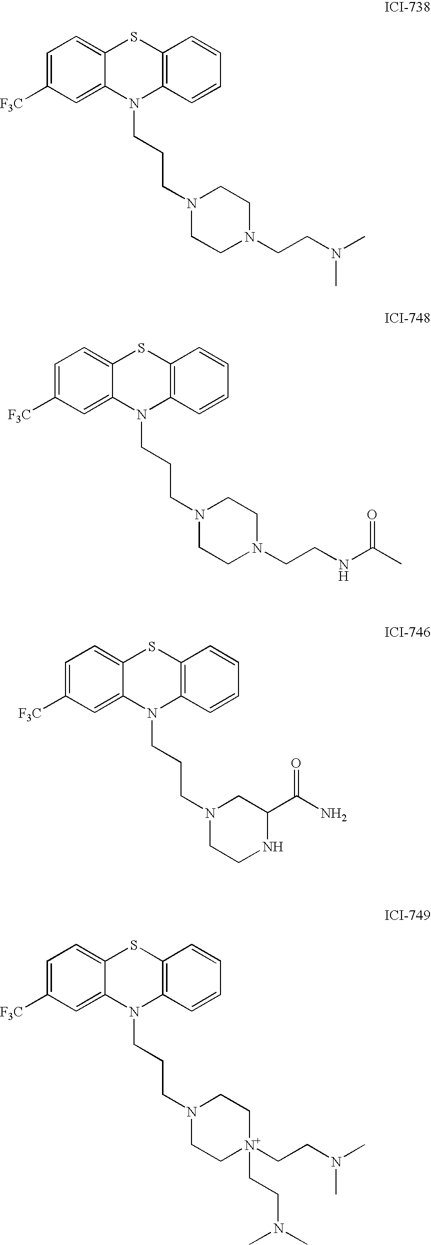

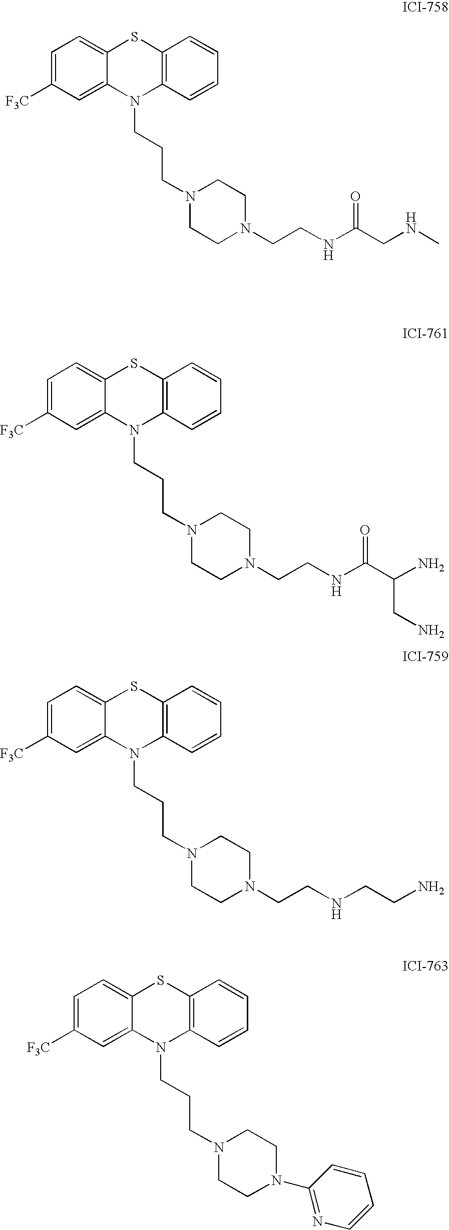

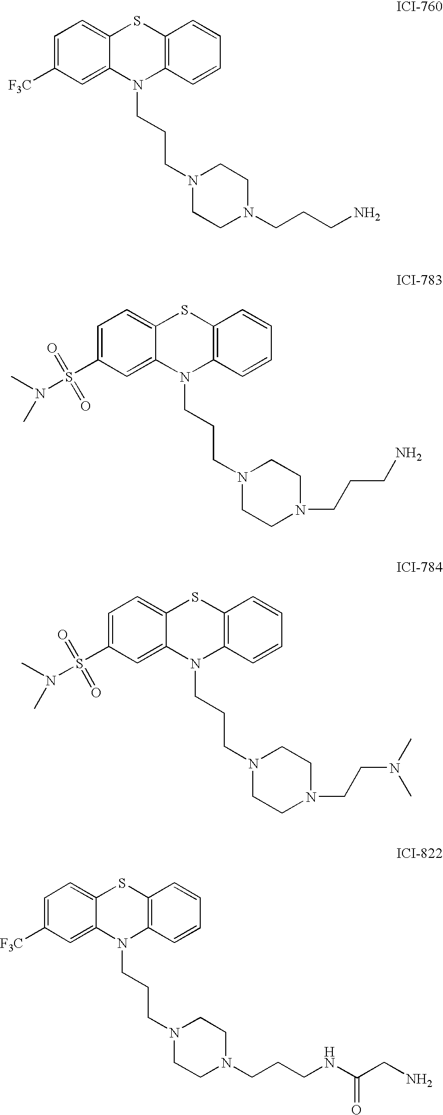

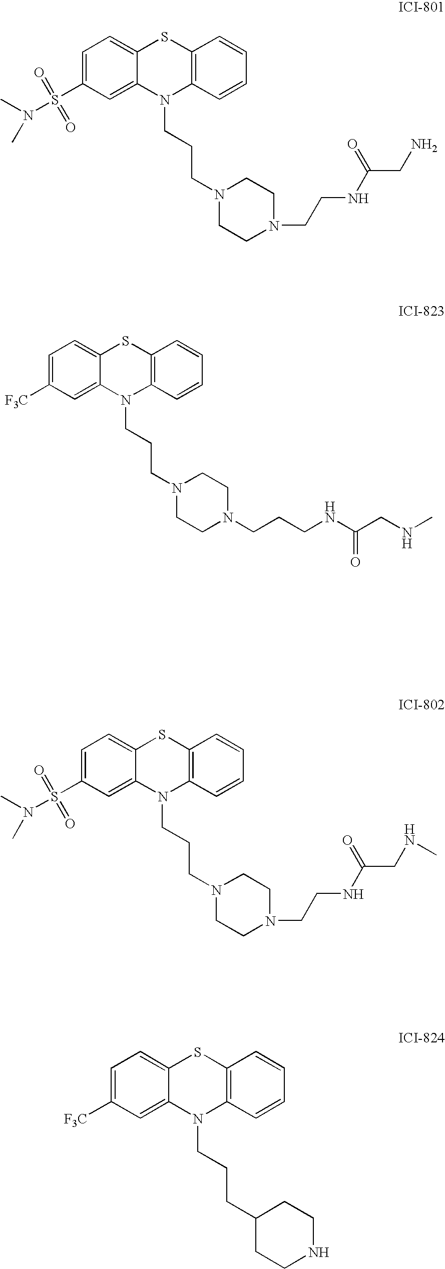

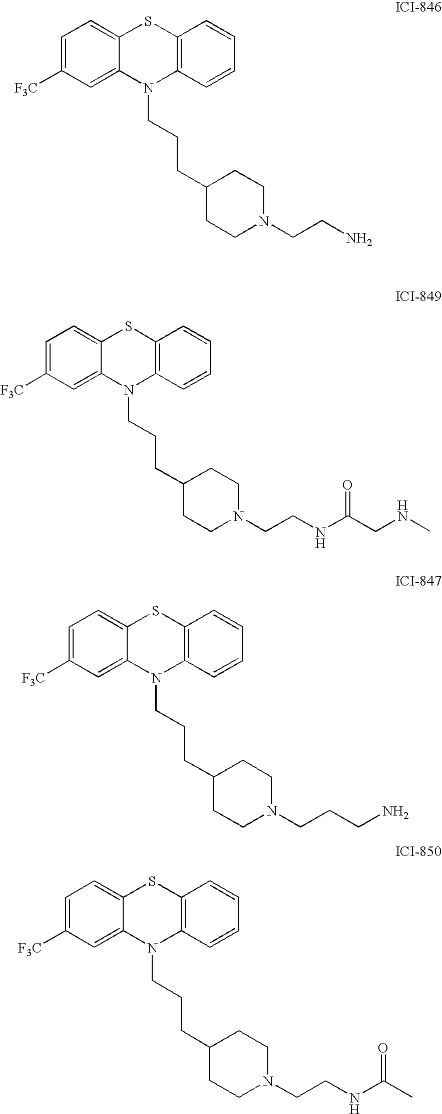

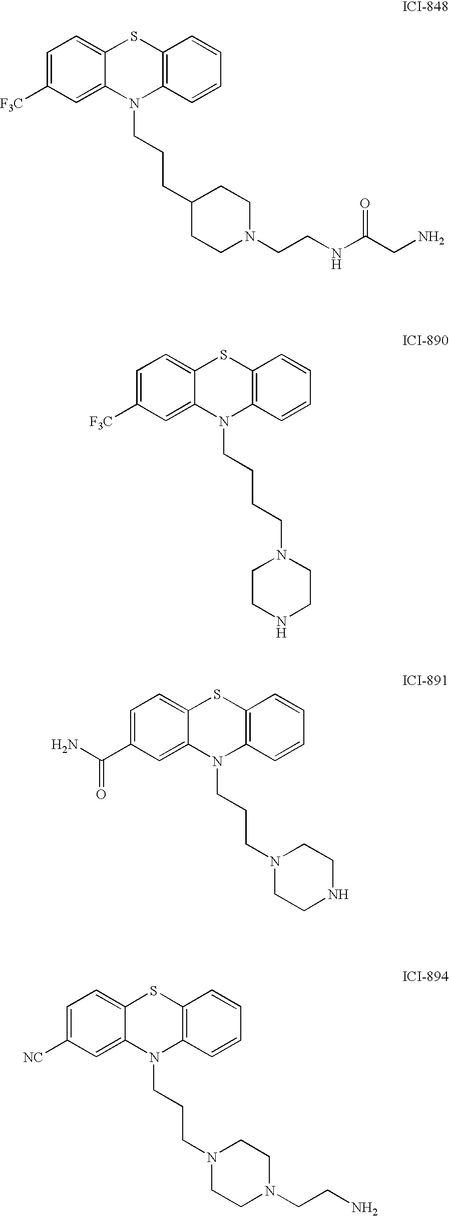

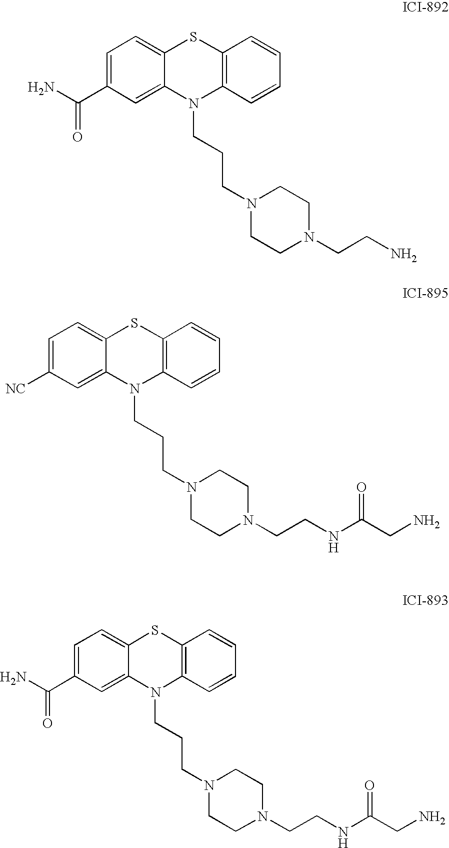

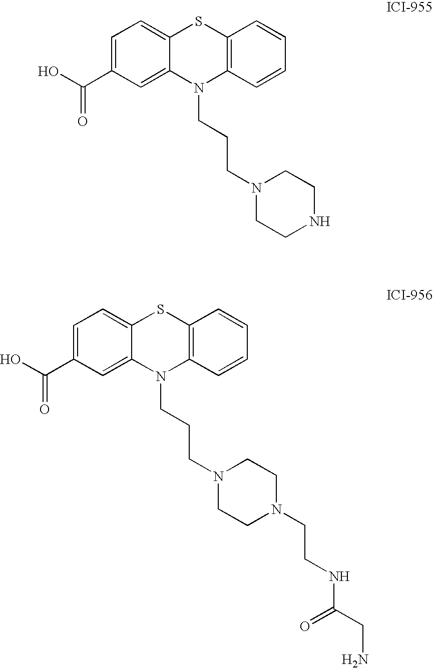

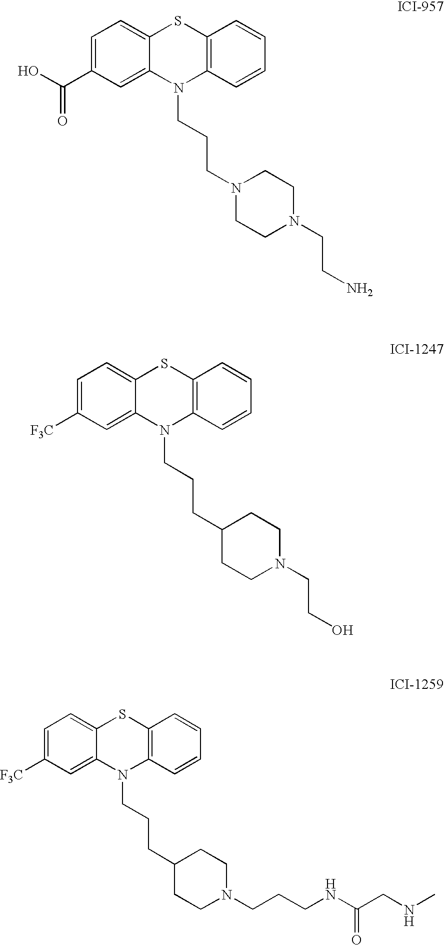

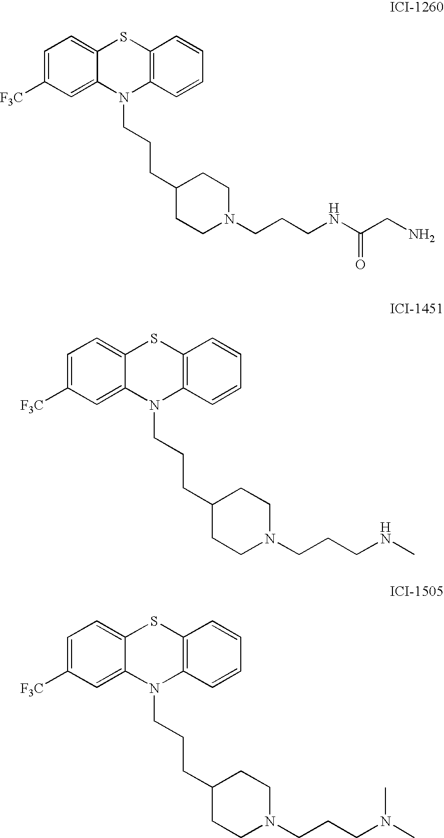

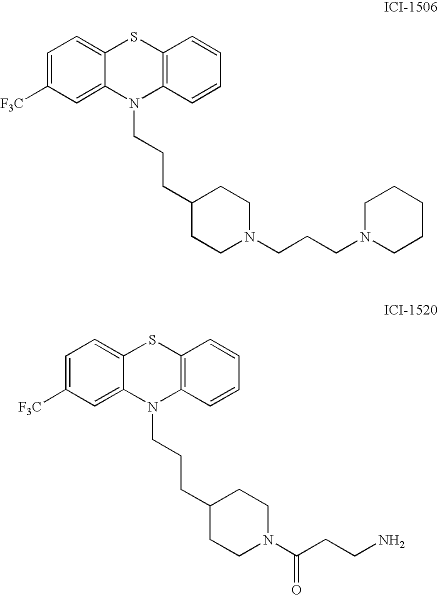

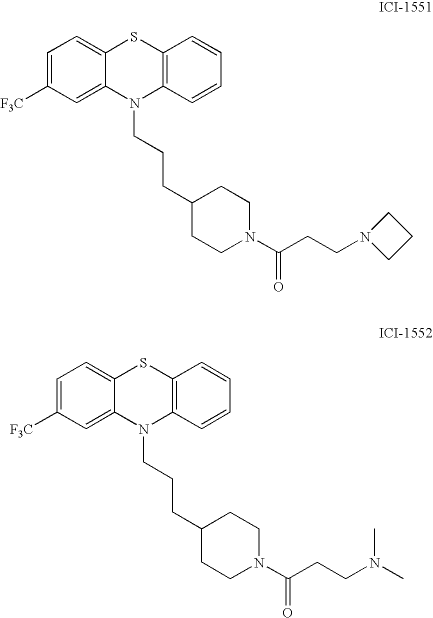

B. 5HT4 Receptor Ligand

Serotonin family receptors play a significant role in the neurological system and as disclosed in the present invention, in the immune system. The data disclosed herein demonstrate that effecting the activation of serotonin type 4 receptors can modulate the immune response. More specifically, inhibition of binding of serotonin with a serotonin type 4 receptor mediates a decrease or inhibition of T cell activation and, among other things, inhibition of both primary and secondary T cell responses in a mammal. Such inhibition of T cell responses provides a powerful therapeutic method for treatment of, inter alia, autoimmune disease and allogeneic graft rejection. A non-limiting exemplary autoimmune disease that can be treated with a 5-HT4 receptor ligand is rheumatoid arthritis.

The invention provides a method (also referred to herein as “screening assays”) for identifying ligand that can be used for the treatment of a disease or condition of the immune system. The methods include the identification of ligands that bind to 5-HT4 receptor and can modulate the immune system. In some instances, the method includes the identification of ligands that bind to 5-HT4 receptor and/or have a stimulatory or inhibitory effect on the biological activity of 5-HT4 receptor or its expression, and then determining which of the ligands have an effect on symptoms or diseases in an in vivo assay. In one embodiment, the present invention includes the use of a ligand for 5-HT4 receptors for the preparation of a pharmaceutical composition for the treatment of a disease or condition of the immune system including, but not limited to, inflammatory and autoimmune diseases, such as multiple sclerosis, Crohn's Disease, rheumatoid arthritis and asthma.

Candidate or test ligands that bind to 5-HT4 receptor and/or have a stimulatory or inhibitory effect on the activity, or the expression of 5-HT4 receptor are identified either in assays that employ cells expressing 5-HT4 receptor on the cell surface (cell-based assays) or in assays with isolated 5-HT4 receptor (cell-free assays). The various assays can employ a variety of variants of 5-HT4 receptor (e.g., full-length 5-HT4 receptor, a biologically active fragment of 5-HT4 receptor, splice variants, or a fusion protein that includes all or a portion of 5-HT4 receptor). Moreover, 5-HT4 receptor can be derived from any suitable mammalian species (e.g., human 5-HT4 receptor, rat 5-HT4 receptor, or mouse 5-HT4 receptor). The assay can be a binding assay entailing direct or indirect measurement of the binding of a test ligand or a known 5-HT4 ligand to 5-HT4 receptor. The assay can also be an activity assay entailing direct or indirect measurement of the activity of 5-HT4 receptor. The assay can also be an expression assay entailing direct or indirect measurement of the expression of 5-HT4 receptor mRNA or 5-HT4 protein. The various screening assays are combined with an in vivo assay entailing measuring the effect of the test ligand on the symptoms of an disease or condition associated with the immune system including, but not limited to, inflammatory and autoimmune diseases, such as multiple sclerosis, Crohn's Disease, rheumatoid arthritis and asthma.

Binding is understood as meaning any molecular interaction between the ligand and the receptor, in particular under physiological conditions. Conventional interactions between a ligand and the corresponding receptor include, but are not limited to electrostatic attraction, hydrogen bonding, hydrophobic bonds, van-der-Waals forces, and metal complex-like coordinative bonds. In addition to the above-mentioned, reversible molecular interactions, irreversible interactions between ligand and receptor can also be possible, such as, for example, covalent bonds.

According to one embodiment, compounds of the present invention include competitive ligands that can competitively inhibit the binding of a natural ligand such as 5-HT (5-hydroxytryptamine) to the appropriate 5-HT4 receptor. In a non-limiting example, a competitive binding format comprises contacting 5-HT4 receptor expressing cell with a known ligand that binds to 5-HT4 receptor to form an assay mixture, contacting the assay mixture with a test ligand, and determining the ability of the test ligand to interact with the 5-HT4 receptor expressing cell, wherein determining the ability of the test ligand to interact with the 5-HT4 receptor expressing cell comprises determining the ability of the test ligand to preferentially bind the 5-HT4 receptor expressing cell as compared to the known ligand.

Competitive inhibition is understood as meaning that ligands of the invention can compete with a comparison ligand, for example, 5-HT or a natural ligand, for binding to the receptor. That is, the binding of one ligand prevents the binding of the other ligand to the same receptor.

According to a further embodiment, ligands of the invention can inhibit the binding of comparison ligands, such as 5-HT or a natural ligand to 5-HT4 receptors, noncompetitively. Noncompetitive inhibition is understood as meaning that ligands of the invention can modulate, via their binding to the receptor, the binding of a comparison ligand to the corresponding receptor.

At least in the case of competitive inhibition, i.e., of reversible binding, the principle applies that the displacement of one ligand by another increases with decreasing binding affinity of the one or increasing binding affinity of the other with respect to the receptor. More expediently, therefore, ligands that can be used according to the invention have a high binding affinity for 5-HT4 receptor. A binding affinity of this type allows, on the one hand, an effective displacement of naturally occurring ligands for 5-HT4 receptor where the necessary concentration of ligand that can be used according to the invention for the binding of a certain amount of this ligand to 5-HT4 receptor decreases with increasing binding affinity. With respect to medical use, ligands are therefore preferred whose binding affinity is so great that these can be administered in justifiable amounts in the course of an effective medical treatment as an active compound. Ligands of the invention are therefore preferably administered in daily doses of approximately 0.01 to 100 mg/kg of body weight and in particular of approximately 0.1 to 15 mg/kg of body weight on parenteral administration and 1 to 30 mg/kg of body weight on oral administration.

The competition experiments referred to above can be used to identify desirable ligand by determining the concentration at which the ligand displaces 50% of another comparison ligand from the receptor binding site (IC₅₀ values). Ligands that have half-maximal inhibition constants IC₅₀ of less than about 10⁻⁵ M are preferred, preferably of less than about 10⁻⁶ M, advantageously of less than about 10⁻⁷ M and in particular of less than about 10⁻⁸ M.

The binding affinity of ligands which can be used according to the invention can also be expressed by means of the inhibition constant Kᵢ, which is in general likewise determined in vitro using competition experiments. For the binding of 5-HT4 receptors, ligands, which can be used according to the invention, preferably have Kᵢ values of less than about 10⁻⁵ M, preferably of less than about 10⁻⁶ M, advantageously of less than about 10⁻⁷ M and particularly preferably of less than about 10⁻⁸ M. Kᵢ values of compounds which can be used according to the invention lie.

Ligands of the invention can bind with a lower, an essentially identical, or a higher affinity to 5-HT4 receptor than to a specific receptor that is different from 5-HT4 receptor. Thus ligands for 5-HT4 receptors with respect to the use according to the invention in particular include those whose binding affinity for 5-HT4 receptors compared with the affinity for 5-HT2 receptors, in particular 5-HT2A, 5-HT2B and/or 5-HT2C, 5-HT6, and 5-HT7 receptors is so high that they are advantageously suitable for the use according to the invention. This does not necessarily presuppose a comparatively more selective binding to 5-HT4 receptors, even though selective ligands for 5-HT4 receptors are a particular embodiment of the present invention. For example, ligands can be used that have high affinity for both 5-HT4 and 5-HT2B receptors.

Selective ligands of the invention have higher binding affinities to 5-HT4 receptors than to one or more 5-HT receptors that are different from 5-HT4 receptor, i.e., in particular receptors allocated to the abovementioned 5-HT receptor classes 5-HT1, 5-HT2, 5-HT3, 5-HT5, 5-HT6 and 5-HT7 receptors. If the binding affinity for 5-HT4 receptor of a ligand is at least two times greater than that of a 5-HT receptor which is different from 5-HT4 receptor, it is referred to as a selective binding ligand to 5-HT4 receptor in relationship to the 5-HT receptor which is different from 5-HT4 receptor. In some instances, the ligands useful for the invention are ligands whose binding affinity for 5-HT4 receptors is at least two times greater than for at least one 5-HT receptor, in particular for 5-HT2A, 5-HT2C, 5-HT6 and/or 5-HT7 receptors.

Selectivity is understood as meaning the property of a ligand to bind preferably to 5-HT4 receptors. In some instances, the preferred ligands of the invention selectively bind to 5-HT4 and 5-HT2B receptors. In other instances, the ligands of the invention selectively bind at least to 5-HT4 and 5-HT2B receptors. In another instance, the ligands of the invention selectively bind at least to 5-HT4,5-HT2B, and 5-HT6 receptors. In yet other instances, the ligands of the invention bind at least to 5-HT4, 5-HT2B, 5-HT6, and 5-HT7 receptors.

It is decisive for the selectivity outlined above that the binding affinities for 5-HT4 receptors on the one hand and for one or more of 5-HT receptors that are different from 5-HT4 on the other hand are adequately different. Affinity differences are preferred according to which binding affinity ratios of at least 2, advantageously of at least 5, particularly advantageously of 10 at least 10, preferably of at least 20, particularly preferably of at least 50 and in particular of at least 100 are present.

The binding of the ligands to 5-HT4 receptor is coupled to an effector function. In some instances, the ligands can act agonistically or antagonistically.

Agonists are designated as ligands according to the invention to imitate the activity of 5-HT or a natural ligand of 5-HT4,5-HT2B, 5-HT6, and/or 5-HT7 receptors.

Antagonists are designated as ligands according to the invention to block the agonistic activity of 5-HT or a natural ligand on 5-HT4,5-HT2B, 5-HT6, and/or 5-HT7 receptors.

According to a preferred embodiment of the present invention, ligands are employed whose binding at least to 5-HT4 receptors bring about a change in the agonist-induced stimulation of GTP binding to membrane-bound G proteins, a change in intracellular calcium levels, a change in agonist-induced induction of phospholipase C activity and/or a change in CAMP production. In a preferred embodiment, use of a ligand to bind at least a 5HT-4 receptor provides a therapeutic effect with respect to an immune reaction such as alleviating symptoms of rheumatoid arthritis. This embodiment also includes ligands that are active in known animal models for rheumatoid arthritis processes.

The use according to the invention is not restricted to the abovementioned ligands. Rather, any substance that binds to 5-HT4 receptors in the manner described above, can be used according to the invention as a 5-HT4 ligand. However, the invention should not be limited to ligands that bind 5-HT4 receptors. This is because 5-HT4 receptor is a seven transmembrane G protein coupled receptor (GPCR) and therefore the ligands of the present invention are capable of binding to other GPCRs, including, but not limited to, receptors for hormones, neurotransmitters, growth factors, dopamine, calcitonin, adrenergic hormones, endothelin, cAMP, adenosine, acetylcholine, serotonin, histamine, thrombin, kinin, follicle stimulating hormone, opsins, endothelial differentiation gene-1, rhodopsins, odorants, cytomegalovirus, phospholipase C, adenyl cyclase, phosphodiesterase, protein kinase A, protein kinase C, and the like.

One skilled in the art when armed with the present disclosure would understand that the binding of a ligand of the invention to a 5-HT4 receptor can allosterically modulate, e.g., allosterically potentiate/enhance or suppress/attenuate, the ability of the 5-HT4 receptor to be bound by other binding site ligands. Thus, it is contemplated that the ligands of the invention can behave as allosteric modulators of 5-HT4 receptor.

Assays for the determination of binding affinities of test substances for 5-HT4 receptors are known in principle. This can be carried out, for example, by assessing the competitive inhibition of the binding of a comparison ligand to 5-HT4 receptors by the substance to be investigated. Suitable comparison ligands are known ligands for 5-HT receptors, such as 5-HT. The binding to ligands to be investigated can also be determined directly on 5-HT receptors. The inhibition constants Kᵢ expressing binding affinity can be determined, for example, calorimetrically, i.e., by measurement of the binding energy released.

Effector functions can also be assessed qualitatively or quantitatively both in vitro and in vivo with the aid of known functional assays. The assessment of an agonistic and/or antagonistic activity can be based on all those effects that are produced by the binding of 5-HT to 5-HT4 receptors. It is preferred according to the invention to assess the effects on the binding of GTP to G proteins, on intercellular calcium levels, on the phospholipase C activity and/or on the cAMP production. These processes are preferably used for secondary screening. The effector functions can also be assessed in an animal model to determine the therapeutic effects of the ligands of the invention to ameliorate the disease state of the animal model.

The effector function can also be assessed for the activity of the ligands of the invention to bind other 5-HT receptors including, but are not limited to 5-HT4, 5-HT2B, 5-HT6, or 5-HT7 receptors. This expediently takes place taking into account the binding affinities determined for 5-HT4 and other 5-HT receptors, i.e., in particular taking into account the selectivity. In some instances, the effector function is associated with binding to both 5-HT4 and 5-HT2B receptors. In other instances, the effector function of the ligands of the invention is associated with the ability for the compound to bind 5-HT4,5-HT2B, 5-HT6,5-HT7 receptors or any combinations thereof.

The present invention therefore also relates to processes for the identification and characterization of ligands which can be used according to the invention. These and further processes, which are similarly suitable, can form the basis for in vitro screening processes with which it is possible from a large number of different ligands to pick out those which, with respect to future use, appear to be the most promising. For example, by means of combinatorial chemistry, extensive substance banks can be prepared which comprise myriads of potential active compounds.

A process according to the invention is used for the determination of the affinity and/or selectivity of ligands for 5-HT4 receptors. For this purpose, the ligand is brought into contact with the 5-HT4 receptor and the binding affinity is determined. For the determination of selectivity, the binding affinity to other 5-HT receptors of the ligand to be investigated is determined in the same manner.

A further process according to the invention relates to the determination for the activity of the ligands on 5-HT4 receptors, i.e. the determination of agonistic, partly agonistic, antagonistic and/or partly antagonistic action. For this purpose, the ligand is brought into contact with a 5-HT4 receptor and the effect caused by the binding is assessed.

### II. Methods

The present invention includes a method of inducing cell death in an immune cell. The method comprises inhibiting the interaction of serotonin with a serotonin receptor by contacting an immune cell with a 5-HT receptor ligand, such as the 5-HT receptor ligand of formula I or a 5-HT receptor ligand disclosed elsewhere herein. More preferably, the 5-HT receptor ligand is selected from, among others, ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, and ICI-1505. This is because, as demonstrated by the data disclosed herein, contacting an immune cell with a 5-HT receptor ligand of the present invention results in, among other things, an inhibition of proliferation of a variety of lymphocytes, including T-cells and B-cells. In addition, the data disclosed herein demonstrates that contacting an immune cell with a 5-HT receptor ligand of the present application results in cell death of the lymphocyte in a dose and time dependent manor. Thus, the present invention comprises inducing cell death in an immune cell and a method of inhibiting proliferation of an immune cell by contacting the lymphocyte with a 5-HT receptor ligand. Preferably, the 5-HT receptor ligand binds to 5-HT4 receptor. In other preferred embodiments, the 5-HT receptor ligand binds at least to both 5-HT4 and 5-HT2B receptors. In other embodiments, the ligand binds at least to 5-HT4,5-HT2B, and 5-HT6 receptors. In yet other embodiments, the ligand binds at least to 5-HT4,5-HT2B, 5-HT6, and 5-HT7 receptors.

The present invention further comprises a method of treating a mammal, preferably a human, having a disease characterized by abnormal lymphocyte proliferation where inhibiting lymphocyte proliferation or inducing cell death in the abnormally proliferating lymphocytes results in treatment of the disease. The method comprises administering an effective amount of a 5-HT receptor ligand to a mammal, preferably a human, in need thereof. As demonstrated by the data disclosed herein, administration of a 5-HT receptor ligand of the present invention results in, among other things, a rapid cessation of proliferation of various types of lymphocytes, including, but not limited to, T-cells and B-cells. In addition, according to the data presented herein, administration of a 5-HT receptor ligand of the present invention results in cell death in the lymphocyte. Inducing cell death or inhibiting proliferation of an immune cell prevents or treats the generation of an immune response, such as those common to inflammatory diseases, autoimmune diseases and transplant rejection, and also treats lymphatic neoplasias, including lymphomas and myelomas.

One of skill in the art would also appreciate, based upon the disclosure provided herein, that the invention encompasses using a 5-HT receptor ligand that is water soluble and that does not substantially cross the blood-brain barrier. This is because one skilled in the art would understand that because serotonin receptors are found on neural cells and, as now disclosed, on cells of the immune system, including tumors derived from such cells (e.g., multiple myelomas, and the like), it is desirable, but not necessary, to inhibit signaling via serotonin receptor on an immune cell while not affecting serotonin signaling via a serotonin receptor on a neural cell. In such instances, administering a compound that inhibits signaling but does not cross the blood-brain barrier where it would affect serotonin signaling in neural cells is desirable.

Accordingly, the present invention encompasses using a compound that while inhibiting serotonin signaling via a serotonin receptor on a cell, does not substantially cross the blood-brain barrier. Such compounds are disclosed elsewhere herein and include the 5-HT receptor ligand of formula I, as well as those disclosed elsewhere herein, but preferably includes ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, and ICI-1505. One skilled in the art would understand, based upon the disclosure provided herein, that methods to modify a compound to affect its ability to cross the blood-brain barrier are well-known in the art, which also teaches a wide plethora of assays for assessing the ability of a substance to cross the barrier. One such method is disclosed herein, i.e., adding various side groups to a compound such as fluphenazine, thereby decreasing the ability of the compounds of the invention to cross the blood-brain barrier. The modified fluphenazine compounds, designated, e.g., formula I, are disclosed herein, but the present application is in no way limited to these or any other particular derivatives of fluphenazine. Instead, the invention encompasses any compound having the desired immunomodulatory characteristics of the inhibitors of the invention, while also possessing the desired reduced ability to cross the blood-brain barrier. The production and identification of compounds having these characteristics are routine in the art, as are assays for assessing the permeability of a compound through the blood-brain barrier. Such assays are exemplified herein, as are methods of producing compounds of interest having the desired characteristics. Nonetheless, the present invention is in no way limited to these, or any other, methods in particular; rather, it includes methods of producing and identifying compounds that do not substantially cross the blood-brain barrier and still inhibit serotonin signaling via a serotonin receptor such as those disclosed herein, known in the art, or to be developed in the future.

The present invention can be used to treat a variety of inflammatory diseases. Inflammatory diseases comprise diseases triggered by cellular or non-cellular mediators of the immune system or tissues causing the inflammation of body tissues and subsequently producing an acute or chronic inflammatory condition. Examples for such inflammatory diseases are hypersensitivity reactions of type I-IV, for example but not limited to hypersensitivity diseases of the lung including asthma, atopic diseases, allergic rhinitis or conjunctivitis, angioedema of the lids, hereditary angioedema, antireceptor hypersensitivity reactions and autoimmune diseases, Hashimoto's thyroiditis, systemic lupus erythematosus, Goodpasture's syndrome, pemphigus, myasthenia gravis, Grave's and Raynaud's disease, type B insulin-resistant diabetes, type 1 insulin-resistant diabetes, rheumatoid arthritis, psoriasis, Crohn's disease, scleroderma, mixed connective tissue disease, polymyositis, sarcoidosis, glomerulonephritis, acute or chronic host versus graft reactions.

The present invention can be used to treat a variety of autoimmune diseases, including, but not limited to, myasthenia gravis, idiopathic inflammatory myopathy, chronic neutropenia, rheumatoid arthritis, idiopathic thromcytopenia purpura, autoimmune hemolytic syndromes, antiphospholipid antibody syndromes, inflammatory bowel disease, Crohn's disease, ulcerative colitis, myocarditis, Guillian-Barre Syndrome, vasculitis, multiple sclerosis, neuromyelitis optica (devic's syndrome), lymphocytic hypophysitis, Graves disease, Addison's disease, hypoparathroidism, type 1 diabetes, systemic lupus erythematosus, pemphigus vulgaris, bullous pemphigoid, psoriasis, psoriatic arthritis, endometriosis, autoimmune orchitis, dystrophic epidermolysis, sarcoidosis, Wegener's granulomatosis, autoimmune deafness, Sjögren's disease, autoimmune uveoretinitis, interstitial cystitis, Goodpasture's syndrome, and fibromyalgia. This is because, as demonstrated by the data disclosed herein, the 5-HT receptor ligands of the present invention inhibit the proliferation of T cells and in some instances both T cells and B cells, and additionally induce cell death in such lymphocytes. Thus, the methods of the present invention comprise administering an effective amount of a 5-HT receptor ligand to a mammal, preferably a human, having an autoimmune disease, e.g. psoriasis.

The invention further comprises compounds and methods for treating asthma. Asthma is believed to arise as a result of interactions between multiple genetic and environmental factors and is characterized by three major features: 1) intermittent and reversible airway obstruction caused by bronchoconstriction, increased mucus production, and thickening of the walls of the airways that leads to a narrowing of the airways, 2) airway hyperresponsiveness, and 3) airway inflammation. Certain cells are critical to the inflammatory reaction of asthma and they include T cells and antigen presenting cells, B cells that produce IgE, and mast cells, basophils, eosinophils, and other cells that bind IgE. These effector cells accumulate at the site of allergic reaction in the airways and release toxic products that contribute to the acute pathology and eventually to tissue destruction related to the disorder. Other resident cells, such as smooth muscle cells, lung epithelial cells, mucus-producing cells, and nerve cells may also be abnormal in individuals with asthma and may contribute to its pathology. While the airway obstruction of asthma, presenting clinically as an intermittent wheeze and shortness of breath, is generally the most pressing symptom of the disease requiring immediate treatment, the inflammation and tissue destruction associated with the disease can lead to irreversible changes that eventually make asthma a chronic and disabling disorder requiring long-term management.

Chronic obstructive pulmonary (or airways) disease (COPD) is a condition defined physiologically as airflow obstruction that generally results from a mixture of emphysema and peripheral airway obstruction due to chronic bronchitis. Emphysema is characterized by destruction of alveolar walls leading to abnormal enlargement of the air spaces of the lung. Chronic bronchitis is defined clinically as the presence of chronic productive cough for three months in each of two successive years. In COPD, airflow obstruction is usually progressive and is only partially reversible. By far the most important risk factor for development of COPD is cigarette smoking, although the disease does also occur in non-smokers.

The invention includes the use of the compounds of the invention in combination with other suitable therapies or therapeutic agents (e.g., rheumatoid arthritis medicament). The therapeutic agent can be administered simultaneously, prior to, or after administration of the compounds of the invention to a mammal in need thereof.

A “rheumatoid arthritis medicament” as used herein is a composition of matter that reduces the symptoms, inhibits the rheumatoid arthritis reaction, or prevents the development of a rheumatoid arthritis reaction. Accordingly, the compounds of the invention can be used in combination with existing rheumatoid arthritis medicaments such as the non-limiting examples disclosed herein. However, the invention should not be limited to only these rheumatoid arthritis medicaments, but rather any suitable therapeutic agent. Use of the combination provides an improved sustained pharmacologic effect that translates to an improved disease management. For instance, the efficacy of the combination of the compounds of the invention with a rheumatoid arthritis medicament is improved over the use of each of the medicaments alone. In some instances, the combination of the compounds of the invention with a rheumatoid arthritis medicament works in a synergistic manner.

Rheumatoid arthritis (RA) is a chronic, systemic autoimmune disorder that causes inflammation and tissue damage in joints and tendon sheaths. It can also produce diffuse inflammation (in the lungs, pericardium, pleura, and the sclera of the eye) and also nodular lesions, most commonly in subcutaneous tissue. Anemia is often associated with the disease. RA can be disabling and painful, with loss of function and mobility. It is diagnosed chiefly on symptoms and signs, but also with blood tests (especially a test called rheumatoid factor) and X-rays.

Various treatments are available. Non-pharmacological treatment includes physical therapy and occupational therapy. Analgesia (painkillers) and anti-inflammatory drugs, as well as steroids, are used to suppress the symptoms, while disease-modifying antirheumatic drugs (DMARDs) are often required to inhibit or halt the underlying immune process and prevent long-term damage. In recent times, a newer group of biologics has increased treatment options.

There is no proven cure for RA, but treatments that minimize symptoms or manage the progression of the disease are available. Pain relievers (analgesics) are commonly used in RA therapy in order to minimize the patient's suffering, but most physicians recommend that treatments include at least one specific anti-rheumatic medication, also named DMARD (disease modifying anti-rheumatic drug), to which other medications and non-medical interventions, such as anti-inflammatory agents, can be added as necessary.

A disease modifying anti-rheumatic drug (DMARD) affects biological measures such as ESR (erythrocyte sedimentation rate—a nonspecific screening test that indirectly measures how much inflammation is in the body) and haemoglobin and autoantibody levels. A DMARD also reduces the rate of damage to bone and cartilage. DMARDs produce durable symptomatic remissions, and delay or halt progression of the disease. This is important as damage caused by RA is usually irreversible. Anti-inflammatories and analgesics improve pain and stiffness but do not prevent joint damage or slow the disease progression.

Since permanent damage to the joints occurs at a very early stage in the disease, it is vital to start the use of DMARD as soon as the disease is diagnosed. Delaying therapy for as little as a few months after the onset of symptoms can result in a worse outcome in the long term. There is therefore considerable interest in establishing the most effective therapy with early arthritis, when the patient is most responsive to therapy.

There are several small-molecule DMARDs in use. Non-limiting examples are azathioprine, ciclosporin (cyclosporine A or cyclosporin), D-penicillamine, gold salts, hydroxychloroquine, leflunomide, methotrexate (MTX), minocycline, sulfasalazine (SSZ), and cyclophosphamide.

Azathioprine (Azasan™/Salix™: US; Imuran™, GlaxoSmithKline: Canada, US, Australia, UK; Azamun™, GlaxoSmithKline: Finland; Imurel™, GlaxoSmithKline: Scandinavia) is an immunosuppressant used in RA, as well as in organ transplantation, inflammatory bowel disease (Crohn's disease and ulcerative colitis) and multiple sclerosis. It is a pro-drug, converted in the body to the active metabolites 6-mercaptopurine and 6-thioinosinic acid. The active metabolites are purine synthesis inhibitors.

Ciclosporin is an immunosuppressant drug widely used in post-allogeneic organ transplant to reduce the risk of organ rejection. Initially isolated from a Norwegian soil sample, ciclosporin, the main form of the drug, is a cyclic nonribosomal peptide of 11 amino acids (an undecapeptide) produced by the fungus Tolypocladium inflatum Gams, and contains D-amino acids, which are rarely encountered in nature. Ciclosporin is also used to treat psoriasis, severe atopic dermatitis and infrequently in rheumatoid arthritis and related diseases, although it is only used in severe cases. It has been investigated for use in many other autoimmune disorders. Ciclosporin is thought to bind to the cytosolic protein cyclophilin (immunophil in) of immunocompetent lymphocytes, especially T-lymphocytes. This complex of ciclosporin and cyclophylin inhibits calcineurin, which under normal circumstances is responsible for activating the transcription of interleukin-2. It also inhibits lymphokine production and interleukin release and therefore leads to a reduced function of effector T-cells. It does not affect cytostatic activity. The drug is marketed as Sandimmune™ (original formulation, Novartis), Neoral™ (a new microemulsion formulation, Novartis), Cicloral™ (Sandoz/Hexyl) and Gengraf™ (Abbott).

D-Penicillamine (Cuprimine™/Depen™) is a metabolite of penicillin, with no antibiotic properties. Penicillamine is used as a form of immunosuppression to treat rheumatoid arthritis. It works by reducing numbers of T-lymphocytes, inhibiting macrophage function, decreasing IL-1, decreasing rheumatoid factor, and preventing collagen from cross-linking.

“Gold salts” are a term used for gold compounds used in medicine. These compounds accumulate slowly in the body and, over time, reduce inflammation, especially related to rheumatoid arthritis, inflammatory bowel disease, psoriatic arthritis, membranous nephritis, lupus erythematosus and, infrequently, juvenile rheumatoid arthritis (JRA). Gold drugs can be administered orally or by intramuscular injection, in which case they are administered weekly for approximately three to five months before less-frequent doses begin. Types of gold salts are: auranofin (Ridaura™, in capsule form for oral administration); sodium aurothiomalate or gold sodium thiomalate (Myocrisin™/Aurolateor™/Myochrysine™, administered by injection); aurothioglucose (Solganal™, administered by injection); sodium aurothiosulfate (gold sodium thiosulfate); and sodium aurothiomalate (gold sodium thiomalate). Regular urine tests to check for protein (indicating kidney damage) and blood tests are needed.

Hydroxychloroquine (Plaquenil™) is an antimalarial drug also used to reduce inflammation in the treatment of rheumatoid arthritis and lupus. The drug is believed to reduce T-lymphocyte transformation and chemotaxis. It may also alter the lysosomal pH in antigen presenting cells, further helping curb inflammation.

Leflunomide (Arava™, Sanofi-Aventis) is a pyrimidine synthesis inhibitor used in active moderate to severe rheumatoid arthritis and psoriatic arthritis. The drug inhibits dihydro-orotate dehydrogenase, an enzyme involved in de novo pyrimidine synthesis. The drug has both antiproliferative and anti-inflammatory effects. This double action is supposed to slow progression of the disease and to cause remission/relief of symptoms of rheumatoid arthritis and psoriatic arthritis such as joint tenderness and decreased joint and general mobility in human patients.

Methotrexate (MTX) is an antimetabolite and antifolate drug used in treatment of cancer and autoimmune diseases. It acts by inhibiting the metabolism of folic acid. It has come into use as a treatment for some autoimmune diseases, including ankylosing spondylitis, Crohn's disease, psoriasis, psoriatic arthritis, rheumatoid arthritis, and scleroderma, along with difficult-to-treat asthma cases.

Minocycline (Minocin™, Lederle) is a member of the broad spectrum tetracycline antibiotics. As an anti-inflammatory used in the treatment of RA, minocycline inhibits apoptosis via attenuation of TNF-alpha, downregulating pro-inflammatory cytokine output. This effect is mediated by a direct action of minocycline on the activated T cells and on microglia, resulting in the decreased ability of T cells to contact microglia. This impairs cytokine production in T cell-microglia signal transduction. Minocycline also inhibits microglial activation, through blockade of NF-kappa B nuclear translocation.

Sulfasalazine (often abbreviated SSZ, Azulfidine™/Salazopyrin™) is a sulfa drug, derived from mesalazine (5-aminosalicylic acid, abbreviated as 5-ASA) and used primarily as an anti-inflammatory agent in the treatment of inflammatory bowel disease as well as for rheumatoid arthritis.

Cyclophosphamide (Endoxan™/Cytoxan™/Neosar™/Revimmune™), also known as cytophosphane, is a nitrogen mustard alkylating agent, used to treat various types of cancer and autoimmune disorders such as RA. It is a “prodrug”, being converted in the liver to active forms that have biological activity. In RA cyclophosphamide works by decreasing the immune system's response, being successful in patients where other DMARDs have been ineffective.

The most important and most common adverse events relate to liver and bone marrow toxicity (MTX, SSZ, leflunomide, azathioprine, gold compounds, D-penicillamine), renal toxicity (cyclosporine A, parenteral gold salts, D-penicillamine), pneumonitis (MTX), allergic skin reactions (gold compounds, SSZ), autoimmunity (D-penicillamine, SSZ, minocycline) and infections (azathioprine, cyclosporine A). Hydroxychloroquine does not affect the bone marrow or liver, and is often considered to be the DMARD with the least toxicity. Unfortunately hydroxychloroquine is not very potent, and is usually insufficient to control symptoms on its own.

DMARDs also include biological agents, generally produced through genetic engineering. Examples of such biological agents are tumor necrosis factor alpha (TNFα) blockers, interleukin 1 blockers, monoclonal antibodies against B cells, anti-CD20 monoclonal antibodies, T cell co-stimulation blockers and interleukin 6 (IL-6) blockers.

Tumor necrosis factor alpha (TNFα, cachexin or cachectin) is a cytokine involved in systemic inflammation and is a member of a group of cytokines that stimulate the acute phase reaction. The roles of TNFα are regulation of immune cells, induction of apoptotic cell death, induction of inflammation, inhibition of tumorigenesis and viral replication. Dysregulation and, in particular, overproduction of TNFα have been implicated in a variety of human diseases, such as RA. TNFα promotes the inflammatory response, which, in turn, causes many of the clinical problems associated with autoimmune disorders such as rheumatoid arthritis, ankylosing spondylitis, Crohn's disease, psoriasis and refractory asthma. These disorders are sometimes treated by using a TNFα inhibitor. This inhibition can be achieved with a monoclonal antibody such as infliximab (Remicade™, Centocor) or adalimumab (Humira™, Abbott), or with a circulating receptor fusion protein such as etanercept (Enbrel™, Amgen/Wyeth).

Certolizumab pegol (Cimzia™, CDP870, UCB), which consists of the pegylated Fab fragment of a humanized anti-TNF monoclonal antibody, is approved for use in Crohn's disease, but has not yet received approval for use in rheumatoid arthritis. In contrast to infliximab and adalimumab, certolizumab does not contain an Fc portion and therefore does not induce complement activation, antibody-dependent cellular cytotoxicity, or apoptosis. The full impact of this structural change on the efficacy of the agent is not clear. Certolizumab is designed to be administered on a monthly basis by subcutaneous administration.

Golimumab (CNTO 148, Centocor) is a human monoclonal antibody that binds TNF. Results of a phase 2 dose-ranging study suggest that patients with active RA despite methotrexate therapy may benefit from golimumab at doses of 50 to 100 mg subcutaneously every two to four weeks (Kay et al., 2008, Arthritis Rheum. 58:964).

Interleukin-1 (IL-1) is a cytokine that induces fever, controls lymphocytes, increases the number of bone marrow cells and causes degeneration of bone joints. IL-1 is actually composed of two distinct proteins, called IL-1α and IL-1β. The pro-inflammatory activity of IL-1 can be neutralized with anakinra (Kineret™, Amgen), an interleukin-1 (IL-1) receptor antagonist. Anakinra blocks the biologic activity of naturally occurring IL-1, including inflammation and cartilage degradation associated with rheumatoid arthritis, by competitively inhibiting the binding of IL-1 to the Interleukin-1 type receptor, which is expressed in many tissues and organs. Anakinra is indicated as monotherapeutic agent or in combination with other disease-modifying agents (DMARDs), other than tumor necrosis factor α (TNFα) blocking agents, for the management of signs and symptoms of rheumatoid arthritis and to inhibit the progression of structural damage associated with the disease in adults with moderately to severely active disease who did not respond appropriately to one or more DMARDs. Anakinra should not be used in combination with etanercept (Enmbrel™), infliximab (Remicade™) or adalimumab (Humira™).

Rituximab (Rituxan™/MabThera™, Biogen/Genentech in US and Roche in Europe) is a chimeric monoclonal antibody against the protein CD20. Rituximab is used in the treatment of B cell non-Hodgkin's lymphoma, B-cell leukemias, and some autoimmune disorders. Rituximab destroys both normal and malignant B lymphocytes, and is therefore used to treat diseases which are characterized by having too many B cells, overactive B cells or dysfunctional B cells. Rituximab has been shown to be an effective rheumatoid arthritis treatment and is now licensed for use in refractory rheumatoid disease. It is approved for use in combination with methotrexate (MTX) for reducing signs and symptoms in adult patients with moderately- to severely-active rheumatoid arthritis (RA) who have had an inadequate response to one or more anti-TNF-α therapies.

Clinical development is reportedly proceeding with fully human anti-CD20 monoclonal antibodies. One such agent is ofatumumab (HuMax-CD20™, Genmab & GlaxoSmithKline), reported in Teeling et al., 2006, J. Immunol. 177:362. Result of a dose ranging study are promising with regard to efficacy, but, like rituximab, infusion reactions (anaphylactoid, urticarial, and bronchospastic) were frequently observed despite pretreatment with acetaminophen and antihistamines.

TRU-015 (Trubion Pharmaceuticals) is a small recombinant anti-CD20 protein with antibody-like properties that depletes B cells by targeting CD20. It reportedly acts via various mechanisms, including: antibody dependent cellular cytotoxicity (ADCC), antibody-dependent complement-mediated cytotoxicity, and apoptotic signaling (Burge et al., 2006, Ann. Rheum. Dis. 65(suppl 2):108). It may have advantages over anti-CD20 antibodies due to its smaller size and consequent better penetration into solid tissues.

The therapeutic response seen with B cell depletion using rituximab suggests that other approaches that interfere with B cell function or interfere with B cell trafficking to the joints or other sites of inflammation in patients with RA may prove useful. Belimumab (LymphoStat-B™, Human Genome Sciences & GlaxoSmithKline) is an anti-BLyS monoclonal antibody (LymphoStat-B) that has been used in a dose ranging, phase II trial in RA. However, the efficacy observed at 24 weeks was rather modest. Although its possible role in the treatment of RA is not known, it may prove to be useful in the future in combination regimens with other B cell targeting agents. Atacicept (TACI-Ig™, ZymoGenetics) is a recombinant fusion protein comprised of a portion of the transmembrane activator and calcium-modulator and an immunoglobulin chain (TACI-Ig or atacicept). Atacicept targets molecules on the B cell surface that promote B cell survival (BLyS and APRIL). Repeated dosing of TACI-Ig produces significant reductions in immunoglobulin levels, including a 41 to 44 percent decrease in rheumatoid factor in the highest dose group (Tak et al., 2008, Arthritis Rheum. 58:61).

Abatacept (Orencia™, BMS) is a fusion protein composed of an immunoglobulin fused to the extracellular domain of CTLA-4, a molecule capable of binding B7, a type of peripheral membrane protein found on activated antigen presenting cells (APCs). Abatacept has a high-affinity binding site for B7, and works by binding to the B7 protein on APCs and preventing them from delivering the costimulatory signal to T cells. This prevents the full activation of T cells. Abatacept is a selective costimulation modulator as it inhibits the costimulation of T cells, and is approved in the US for the treatment of rheumatoid arthritis in the case of inadequate response to anti-TNFα therapy. Belatacept (Bristol Myers Squibb) is a second-generation CTLA4-Ig that has superior binding to CD80 and CD86 compared with abatacept. This medication has been used primarily in organ transplantation so far.

Tocilizumab (Actemra™, Roche and Chugai) is a humanized monoclonal antibody against IL-6R (interleukin-6 receptor) used as an immunosuppressive drug, useful in the treatment of RA. Interleukin-6 (IL-6) is an interleukin that acts as both a pro-inflammatory and anti-inflammatory cytokine.

Antiinflammatory agents are often used in the treatment of RA. Such agents include glucocorticoids and non-steroidal anti-inflammatory drugs (NSAIDs).

Glucocorticoids are a class of steroid hormones characterised by an ability to bind with the glucocorticoid receptor (GR) and trigger similar effects. Glucocorticoids are distinguished from mineralocorticoids and sex steroids by their specific receptors, target cells, and effects. In technical terms, corticosteroid refers to both glucocorticoids and mineralocorticoids (as both are mimics of hormones produced by the adrenal cortex), but is often used as a synonym for glucocorticoid. In this document, glucocorticoid and corticosteroid are used interchangeably.

Corticosteroids are used long-term to prevent development of the symptoms, and suppress, control, and reverse inflammation arising from an initiator. Some corticosteroids can be administered by inhalation and others are administered systemically. The corticosteroids that are inhaled have an anti-inflammatory function by blocking late-reaction allergen and reducing airway hyper-responsiveness. These drugs also inhibit cytokine production, adhesion protein activation, and inflammatory cell migration and activation. They are also believed to reverse β₂-receptor downregulation and to inhibit microvascular leakage.

Corticosteroids include, but are not limited to, beclomethasone dipropionate (inhaler: Becotive™/Qvar™; nasal spray: Beconase™/Vancenase™), budesonide (Rhinocort™/Pulmicort™, AstraZeneca), flunisolide (AeroBid™/Nasaline™/Nasarel™), fluticasone propionate (Flovent™/Flonase™, GlaxoSmithKline; Flixotide™/Flixonase™, Allen & Hanburys), fluticasone furoate (Veramyst™, GlaxoSmithKline), triamcinolone (Kenalog™/Aristocort™/Nasacort™/Tri-Nasal™/Triderm™/Azmacort™/Trilone™/Volon A™/Tristoject™/Fougera™/Tricortone™/Triesence™), and dexamethasone.

Systemic corticosteroids include, but are not limited to, methylprednisolone (Medrol™/Solu-Medrol™, Sandoz), prednisolone (Teva, KV Pharmaceutical) and prednisone (Deltasoneprednisone™, Pharmacia & UpJohn).

Inhaled glucocorticoids are the most widely used prevention medications and normally come as inhaler devices: ciclesonide (Alvesco™, Nycomed), beclomethasone (inhaler: Becotive™/Qvar™; nasal spray: Beconase™/Vancenase™), budesonide (Rhinocort™/Pulmicort™, AstraZeneca), flunisolide (AeroBid™/Nasaline™/Nasarel™), fluticasone (Flovent™/Flonase™/Veramyst™, GlaxoSmithKline; Flixotide™/Flixonase™, Allen & Hanburys), mometasone (Nasonex™/Asmanex Twisthaler™, Schering-Plough), and triamcinolone (Kenalog™/Aristocort™/Nasacort™/Tri-Nasal™/Triderm™/Azmacort™/Trilone™/Volon A™/Tristoject™/Fougera™/Tricortone™/Triesence™).

Non-steroidal anti-inflammatory drugs (NSAIDs) have analgesic, antipyretic and, in higher doses, anti-inflammatory effects—they reduce pain, fever and inflammation. Most NSAIDs act as non-selective inhibitors of the enzyme cyclooxygenase, inhibiting both the cyclooxygenase-1 (COX-1) and cyclooxygenase-2 (COX-2) isoenzymes. This prevents formation of prostaglandins and thromboxane, interfering with the process of inflammation. NSAIDs can be broadly classified based on their chemical structure. Examples of chemical series of NSAIDs are salicylates, arylalkanoic acids, 2-arylpropionic acids, N-arylanthranilic acids, pyrazolidines, oxicams, COX-2 inhibitors, and sulphonanilides.

Examples of salicylate NSAIDs are acetylsalicylic acid (Aspirin), amoxiprin, benorylate/benorilate, choline magnesium salicylate, diflunisal (Dolobid™, Merck), ethenzamide, faislamine, methyl salicylate (oil of wintergreen), magnesium salicylate, salicin, salicyl salicylate, and salicylamide.

Examples of arylalkanoic acid NSAIDs are diclofenac (Voltaren™/Flector™ patch/Diclon™/Panamor™/Arthrotec™/Dedolor™/Deflamat™/Zolterol™), aceclofenac (Preservex™, UCB Pharma), acemethacin, alclofenac, bromfenac (Duract™), etodolac (Lodine SR™, Shire; Eccoxolac™, Meda), indomethacin (Indocin™/Indochron ER™/Indocin SR™), nabumetone (Relafen™/Relifex™/Gambaran™), oxametacin, proglumetacin (Afloxan™/Protaxon™/Proxil™), sulindac (Clinoril™, Merck) and tolmetin (Tolectin™).

Examples of 2-arylpropionic acid (profen) NSAIDs are ibuprofen (Nurofen™/Advil™/Motrin™), alminoprofen, benoxaprofen (Oraflex™, Eli Lilly), carprofen (Rimadyl™, Pfizer), dexibuprofen, dexketoprofen (Keral™, Manarini), fenbufen (Cepal™/Cinopal™/Cybufemm/Lederfen™/Reugast™), fenoprofen (Fenopron™, Typharm), flunoxaprofen, flurbiprofen (Ansaid™, Pfizer; Froben™, Abbott), ibuproxam, indoprofen, ketoprofen (Orudis KT™/Oruvail™/Actron™), ketorolac (Toradol™/Acular™, Syntex/Allergan), loxoprofen (Loxonin™, Sankyo), naproxen (Naprosyn™/Aleve™/Proxen™), oxaprozin (Daypro™/Duraprox™), pirprofen, suprofen (Profenal™) and tiaprofenic acid (Surgam™/Surgamyl™/Tiaprofen™).

Examples of N-arylanthranilic acid (fenamic acid) NSAIDs are mefenamic acid (Ponstel™/Ponstan™/Ponstal™, Pfizer), flufenamic acid (Achless™/Ansatin™/Arleform/Dignodolin™/Fullsafe™/Meralen™/Paraflum™/Parlef™/Rheuma Lindofluid™/Sastridex™/Surika™/Tecramine™), meclofenamic acid, and tolfenamic acid (Clotam Rapid™/Tufnil™).

Examples of pyrazolidine NSAIDs are phenylbutazone, ampyrone, azapropazone (Rheumox™, Goldshield), clofezone, kebuzone, metamizole (Dipyrone™/Analgin™/Novalgin™/Melubrin™, Sanofi-Aventis), mofebutazone, oxyphenbutazone, phenazone and sulfinpyrazone.

Examples of oxicam NSAIDs are piroxicam (Feldenem, Pfizer), droxicam, lornoxicam, meloxicam (Mobic™, Boehringer Ingelheim) and tenoxicam (Mobiflex™, Roche).

Examples of COX-2 inhibitor NSAIDs are celecoxib (Celebrex™, Pfizer), etoricoxib (Arcoxia™, Merck), lumiracoxib (Prexige™, Novartis), parecoxib (Dynastat™, Pfizer), rofecoxib (ViOxx™, Merck) and valdecoxib (Bextra™, Pfizer).

An example of sulphonanilide NSAIDs is nimesulide (Aulin™/Mesulid™).

Other examples of NSAIDs are licofelone (Merck Pharma GmbH) and omega-3 fatty acids.

Analgesics are also often used in the treatment of RA, with the objective of alleviating the pain suffered by the patients. Such agents include acetaminophen (Tylenol™/Paracetamol™), opiates, diproqualone (an analogue of methaqualone, which is commonly known as Quaaludes™ and a component of Mandrax™) and lidocaine (xylocalne) topical.

Opiates are narcotic alkaloids, including morphine, codeine (methylmorphine), thebaine, hydrocodone (Vicodin™/Hycodan™/Lorcet™/Norco™/Hydrokon™), acetyldihydrocodeine, oxycodone (Eukodal™/Eucodal™/Dinarkon™), oxymorphone (Opana™/Numorphamm/Numorphone™, Endo), buprenorphine (Temgesic™/Buprenex™/Suboxone™/Subutex™) and papaverine.

The present invention also comprises compositions and methods for the treatment of immune-cell related diseases and disorders. In an aspect, the disease or disorder is not autoimmune-related.

The present invention further comprises a method of treating organ transplant rejection in a mammal in need thereof. Specifically contemplated in the present invention are methods of treating graft versus host disease (GVHD) and organ transplant rejection by administering a 5-HT receptor ligand disclosed herein to a patient suffering from GVHD and/or organ transplant rejection. The present invention comprises methods of treating, for example, transplant rejection of thoracic organs, such as heart transplants, lung transplants and en bloc heart/lung transplants. The methods of the invention further comprise treating rejection of abdominal organs, such as liver, kidney, pancreas, small bowel and combined transplants, such as kidney/pancreas transplants, liver/kidney transplants, and combined liver/small bowel transplants. The methods of the present invention further comprise treatment after rejection of a hand, cornea, skin or face transplant. In addition, the methods of the present invention can be used to treat rejection of tissues, cells and fluids that are commonly transplanted, including, but not limited to, pancreatic islet cells (islets of Langerhans), bone marrow transplants, adult stem cell transplants, blood transfusions, blood vessel grafts, heart valve grafts, where autologous, allogenic or xenogenic, and bone grafts. This is because, as demonstrated by the data disclosed herein, administering the 5-HT receptor ligands of the present invention results in inhibited proliferation of T cells, one of the effector cells in transplant and graft rejection, and induces cell death in B cells, which produce anti-graft antibodies. Thus, the invention encompasses a method of treating transplant rejection by administering an effective amount of the 5-HT receptor ligands of the present invention to a mammal, preferably a human, in need thereof.

The methods of the present invention further comprise treating a mammal having an autoimmune disease or a mammal rejecting an organ or tissue transplant with a combination of a 5-HT receptor ligand with another immunomodulatory agent. Such immunomodulatory agents include, but are not limited to, other agents, such as, but not limited to, regulators of gene expression (e.g., steroids and glucocorticoids, alkylating agents which are known mutagens (e.g., cyclophosphamide), inhibitors of kinases and phosphatases that act on the calcineurin and JNK/p38 kinase pathways and the cyclin kinase cascade (e.g., CyclosporinA, Tacrolimus [FK506], and Rapamycin), inhibitors of de novo purine synthesis which act as inhibitors of guanosine nucleotide synthesis and are used to prevent allograft rejection and to treat ongoing rejection (e.g., Mycophenolate motefil), and inhibitors of de novo pyrimidine synthesis which are used to treat patients afflicted with rheumatoid arthritis (e.g., Leflunomide), TNF-α inhibitors, such as Adalimumab, Etanercept, Infliximab, and other immunomodulating agents, such as methotrexate, azathioprine, natalizumab, and mercaptopurine.

The immunomodulatory agents of the present invention can be combined with a 5-HT receptor ligand of the present invention, such as the 5-HT receptor ligand of formula I, ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, or ICI-1505, to treat a patient having an autoimmune disease or a patient experiencing transplant rejection. The immunomodulatory agent can be combined with a 5-HT receptor ligand and delivered as one dose or a series of doses, either together or separately. Methods for the combinations of drugs and dosages are described elsewhere herein.

The present invention further comprises a method of treating neoplasias in a human, preferably lymphomas and myelomas. This is because, as demonstrated by the data disclosed herein, neoplastic lymphoma and myeloma cells, when contacted with a 5-HT receptor ligand of the present invention, cease proliferating. Thus, the present invention comprises methods for treating a mammal, preferably a human, having a lymphoma or a myeloma, the method comprising administering to the mammal an effective amount of a 5-HT receptor ligand of the present invention. Such 5-HT receptor ligands include, but are not limited to the 5-HT receptor ligand of formula I, ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, or ICI-1505.

Additional combination therapies specifically contemplated in the present invention include a 5-HT receptor ligand and dexamethasone with or without thalidomide, a 5-HT receptor ligand and thalidomide, a 5-HT receptor ligand and vincristine, a 5-HT receptor ligand and doxorubicin, a 5-HT receptor ligand and melphalan, and a 5-HT receptor ligand with melphalan and prednisone. In relapsed patients, or patients otherwise not responding to conventional rheumatoid arthritis therapies, the invention encompasses methods of treating rheumatoid arthritis in a patient comprising administering combinations of a 5-HT receptor ligand and cyclophosphamide, a 5-HT receptor ligand and bortezomib, or a 5-HT receptor ligand and lenalidomide.

The combinations of a 5-HT receptor ligand and another rheumatoid arthritis therapy are, as demonstrated by the data disclosed herein, effective at inhibiting proliferation and inducing cell death in rheumatoid arthritis cells. As a non-limiting example, micromolar concentrations of the present 5-HT receptor ligand when used in combination with conventional rheumatoid arthritis therapy are effective in, among other things, increased cell death and decreased proliferation when compared to conventional rheumatoid arthritis therapies alone.

As further demonstrated by the data disclosed herein, the 5-HT receptor ligands of the present invention induce cell death and inhibit proliferation in a variety of lymphocytes, and thus are useful in the treatment of various immune system related diseases. Thus, the present invention further comprises a method of inhibiting an immune response in a mammal, preferably a human, by inhibiting serotonin binding with a serotonin receptor by administering a 5-HT receptor ligand of the present invention, thereby inhibiting an immune reaction by the cell, which in turn inhibits an immune response mediated by that cell. Preferably, the ligand of the invention is capable of binding to 5-HT4 receptor. In other preferred embodiments, the 5-HT receptor ligand binds at least to 5-HT4 and 5-HT2B receptors. In other embodiments, the ligand binds at least to 5-HT4,5-HT2B, and 5-HT6 receptors. In yet other embodiments, the ligand binds at least to 5-HT4,5-HT2B, 5-HT6, and 5-HT7 receptors.

The invention further comprises a method of inhibiting an immune reaction by an immune cell. This is because, as set forth elsewhere herein, inhibition of serotonin binding with a serotonin receptor on the immune cell inhibits activation of the cell, which in turn inhibits an immune reaction by that cell when compared to the immune reaction by that cell in the absence of inhibition of serotonin binding and/or when compared with the immune reaction of an otherwise identical cell wherein serotonin binding with its receptor is not inhibited. The present invention further encompasses a method of inhibiting activation of an immune cell, such as a lymphocyte, in a mammal, preferably, a human, wherein the activation is mediated by activation of a serotonin receptor on the cell. Again, this is because, as more fully set forth elsewhere herein, the data disclosed herein demonstrate that inhibiting serotonin signaling via a serotonin receptor on an immune cell by contacting the cell with a 5-HT receptor ligand inhibits activation of the cell, and therefore, also inhibits the immune response that would otherwise be produced by that cell.

The 5-HT receptor ligand, alone or in combinations described herein, that inhibits the serotonin receptor-mediated signals can be administered to a cell, a tissue, or an animal to inhibit interaction of serotonin with a serotonin type receptor on a cell, a tissue, or in an animal. Methods for the safe and effective administration of the 5-HT receptor ligands described herein are known to those skilled in the art. For instance, the administration of serotonin ligands is described in the standard literature. That is, the administration of many serotonin-affecting agents, serotonin receptor ligands, and fluphenazine is set forth in the Physician's Desk Reference (1996 edition, Medical Economics Co., Montvale, N.J.), the disclosure of which is incorporated by reference as if set forth in its entirety herein.

For administration of a 5-HT receptor ligand of the present invention to a mammal, the compound can be suspended in any pharmaceutically acceptable carrier, for example, sterile water or a buffered aqueous carriers, such as glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., N.J.), the disclosure of which is incorporated by reference as if set forth in its entirety herein.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, bolus injection, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Alternatively, compositions of the invention may be administered to the desired location by iontophoresis, using common iontophoresis technology well known to those of skill in the art. Iontophoresis or ElectroMotive Drug Administration (EMDA) is defined as the topical introduction of ionized drugs into the skin using direct current. Iontophoresis is a very effective method of delivering drugs to the affected site. Instead of injecting the compound directly into the inflamed area, iontophoresis of the compound spreads a high concentration of the compound evenly through the tissue. There are several different devices and methods that are useful for iontophoresis of a compound into the skin of a human. For example, ProMed Products, RA Fischer Company, and others, supply devices for iontophoresis of drugs across the skin. In addition, more recent iontophoresis methods and devices are disclosed in U.S. Pat. Nos. RE38,341, RE38,000, RE37,796 and RE36,626 and U.S. patent application No. 2004/0039328 all to Henley, and are hereby incorporated herein by reference in their entirety.

In yet another embodiment, compositions of the invention may be administered to the desired location of a mammal by a transdermal patch. A transdermal patch is a system capable of delivery of a compound to a mammal via the skin, or any suitable external surface, including mucosal membranes, such as those found inside the mouth. Such delivery systems generally comprise a flexible backing, an adhesive and a compound retaining matrix, the backing protecting the adhesive and matrix and the adhesive holding the whole on the skin of the mammal. On contact with the skin, the compound-retaining matrix delivers the compound to the skin, the compound then passing through the skin into the mammal's system.

Certain embodiments of the invention provide a pharmaceutical preparation/dosage formulation provided in the form of a transdermal patch and formulated for sustained release formulation, in a therapeutically effective amount sufficient to treat a disease associated with activation of an immune cell (e.g., rheumatoid arthritis) in a patient, wherein the dosage formulation, when administered (provided as a patch) to the patient, provides a substantially sustained dose over at least about 2 hours, 4 hours, 6 hours, 8, hours, 12 hours, 20 hours, or at least about 24 hours.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration.

Compounds that are identified using any of the methods described herein may be formulated and administered to a mammal for treatment of immune system conditions (i.e., autoimmune diseases and allograft rejection), are now described.

The invention encompasses the preparation and use of pharmaceutical compositions comprising a compound useful for treatment of a wide variety of disorders such as T cell lymphomas, autoimmune disorders (see infra), complications arising from solid organ transplants, skin graft rejection, graft versus host disease in bone marrow transplants, rheumatoid arthritis, and the like.

The pharmaceutical compositions described herein can be prepared alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, bolus injections, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those that comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 0.1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles that comprise the active ingredient and that have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, and the like. Preferably, the compound is, but need not be, administered intraperitoneally.

Thus, the skilled artisan would appreciate, once armed with the teachings provided herein, that the invention encompasses administration of an inhibitor of the interaction of serotonin with a serotonin receptor, preferably the inhibitor is a 5-HT receptor ligand of formula I, ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, or ICI-1505.

### III. Kits

The invention encompasses various kits relating to binding 5-HT4 receptor with a ligand whereby the ligand does not have a substantial effect on central nervous system function. As disclosed elsewhere herein, the ligands of the invention bind 5-HT4 receptor and are useful for inhibiting activation of an immune cell thereby inhibiting an immune response. Thus, in one aspect, the invention includes a kit for modulating an immune response in a mammal. The kit comprises an effective amount of a ligand of the invention. The kit further comprises an applicator and an instructional material for the use thereof.

Additionally, one skilled in the art would appreciate, based upon the disclosure provided herein, that the ligand can be a compound that does not cross the blood-brain barrier and is preferably water soluble. This is because, as more fully discussed elsewhere herein, it may be desirable to inhibit serotonin signaling in a non-neural cell, while not affecting such signaling in a neural cell, which would be protected beyond the blood-brain barrier.

In a specific embodiment, the kit of the present invention comprises a 5-HT receptor ligand, an applicator, and an instructional material for the use thereof. In another embodiment, the kit can comprise a 5-HT4 receptor ligand, such as those described elsewhere herein, a container holding the 5-HT4 receptor ligand, and an instructional material. The skilled artisan can provide the applicator. In still another embodiment, the kit can comprise a compound that binds to both 5-HT4 and 5-HT2B receptors. In other embodiments, the kit can comprise a compound that binds at least to 5-HT4,5-HT2B, and 5-HT6 receptors. In yet other embodiments, the kit can comprise a compound that binds at least to 5-HT4,5-HT2B, 5-HT6, and 5-HT7 receptors.

Preferably, the kit of the present invention comprises a 5-HT receptor ligand of formula I, ICI-681, ICI-682, ICI-683, ICI-684, ICI-685, ICI-686, ICI-687, ICI-696, ICI-697, ICI-712, ICI-713, ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, ICI-953, ICI-954, ICI-955, ICI-956, ICI-957, ICI-1247, ICI-1259, ICI-1260, ICI-1451, or ICI-1505. Additionally, the kit can comprise an instructional material and an applicator for the administration of a 5-HT receptor ligand of the present invention, preferably a 5-HT4 receptor ligand. In some instances, the ligand of the present invention is able to at least bind to 5-HT4 and 5-HT2B receptors. In other embodiments, the ligand binds at least to 5-HT4,5-HT2B, and 5-HT6 receptors. In yet other embodiments, the ligand binds at least to 5-HT4,5-HT2B, 5-HT6, and 5-HT7 receptors.

The kits of the present invention can be used to treat the diseases and conditions disclosed elsewhere herein. Specifically, the kits of the present invention can be used to treat, among other things, inflammatory diseases, such as rheumatoid arthritis, autoimmune diseases, such as psoriasis, organ transplant rejection, such as kidney transplant rejection, lymphoma, such as Hodgkin's lymphoma or non-Hodgkin's lymphoma, and B-cell neoplasias, such as multiple myeloma. The kits described in the present invention are not limited to the uses above however, and can be used in any method derived from the teachings disclosed herein.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLES

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Example 1

### Selection of Candidate Agents

A criterion for selecting an agent for proceeding to clinical trials is whether the compound crosses the blood-brain barrier. In the experiments discussed herein, it is desirable to select a compound that does not cross the blood-brain barrier. Fluphenazine is used as a control compound that has the potential to cross the blood-brain barrier. Table 1 summarizes the results of the test compounds for their potential to cross the blood-brain barrier. From the tested compounds, all compounds were observed to have a low potential to cross the blood-brain barrier.

TABLE 1
Potential to cross the blood-brain barrier
MDR1-MDCK Permeability

| | % | | Papp (×10−6 | | | Brain |
|---|---|---|---|---|---|---|
| Test | Recovery | | cm/s) | | | Penetration |
| Compound ID # | A-B | B-A | A-B | B-A | Efflux | Classification |
| ICI-00847 | 9 | 31 | 0.56 | 9.56 | 17 | Low |
| ICI-00890 | 15 | 29 | 0.75 | 8.60 | 11 | Low |
| ICI-00894 | 56 | 56 | 0.22 | 30.0 | 136 | Low |
| ICI-00715 | 85 | 77 | 0.08 | 12.3 | 154 | Low |
| ICI-00685 | 17 | 15 | 0.66 | 4.47 | 6.8 | Low |
| ICI-00850 | 11 | 9 | 0.94 | 4.94 | 5.3 | Low |
| ICI-00954 | 69 | 18 | 0.15 | 19.6 | 132 | Low |
| ICI-00953 | 23 | 7 | 0.47 | 5.31 | 11 | Low |
| ICI-00848 | 22 | 21 | 0.24 | 15.5 | 64 | Low |
| ICI-01505 | 3 | 26 | <0.36 | 0.64 | >1.8 | Low |
| ICI-00758 | 59 | 41 | 0.21 | 24.9 | 116 | Low |
| ICI-00735 | 47 | 33 | 0.23 | 28.6 | 124 | Low |
| ICI-01417 | 75 | 79 | 2.05 | 87.7 | 43 | Low |
| ICI-01260 | 55 | 47 | 0.32 | 26.4 | 82 | Low |
| ICI-01175 | 24 | 43 | 8.70 | 9.63 | 1.1 | High |
| ICI-01176 | 35 | 55 | 0.78 | 34.2 | 44 | Low |
| ICI-01008 | 3 | 1 | 0.20 | 0.21 | 1.0 | Low |
| ICI-01259 | 29 | 39 | <0.29 | 12.5 | >43 | Low |
| ICI-00760 | 46 | 40 | <0.25 | 18.8 | >75 | Low |
| ICI-00761 | | | | | | |
| ICI-00824 | 11 | 21 | 1.77 | 13.8 | 7.8 | Low |
| ICI-00846 | 3 | 1 | <0.36 | <0.49 | ND | Low |
| ICI-00849 | 11 | 26 | <1.00 | 5.10 | >5.1 | Low |
| ICI-01007 | 19 | 36 | 0.79 | 16.7 | 21 | Low |
| ICI-00822 | 61 | 22 | 0.13 | 15.7 | 119 | Low |
| ICI-00823 | 57 | 46 | <0.24 | 19.4 | >80 | Low |
| ICI-00738 | 15 | 40 | 1.06 | 3.33 | 3.1 | Low |

Brain Penetrating Potential Classification:
1) A-B Papp > 3.0 and Efflux Ratio < 3.0: High
2) A-B Papp > 3.0 and 10 > Efflux Ratio > 3.0: Moderate
3) A-B Papp > 3.0 and Efflux Ratio > 10: Low
4) A-B Papp < 3.0: Low

The results presented herein demonstrate that adding various side groups to a compound such as Fluphenazine, decreases the ability of the modified Fluphenazine to cross the blood-brain barrier. Without wishing to be bound by any particular theory, modification of a fluphenazine can reduce the ability of the compound to cross the blood-brain barrier.

The next set of experiments was designed to assess compounds that have low levels of brain penetration for the ability of the test compounds to inhibit cellular proliferation. An exemplary in vitro assay is to screen for the ability of the test compounds to inhibit proliferation of the B-cell neoplastic cell line RPMI 8226 (a plasmacytoma derived from a multiple myeloma patient (Matsuoka, et al., 1967, Proc. Soc. Exp. Biol. Med. 125: 1246-1250). However other cell line can be used such as, U266 (established from an IgE-secreting myeloma patient (Nilsson, et al., 1970, Clin. Exp. Immunol., 7: 477-489) and ARH77 (an EBV transformed plasma cell leukemia (Burk, et al., 1978, Cancer Res. 38: 2508-2513).

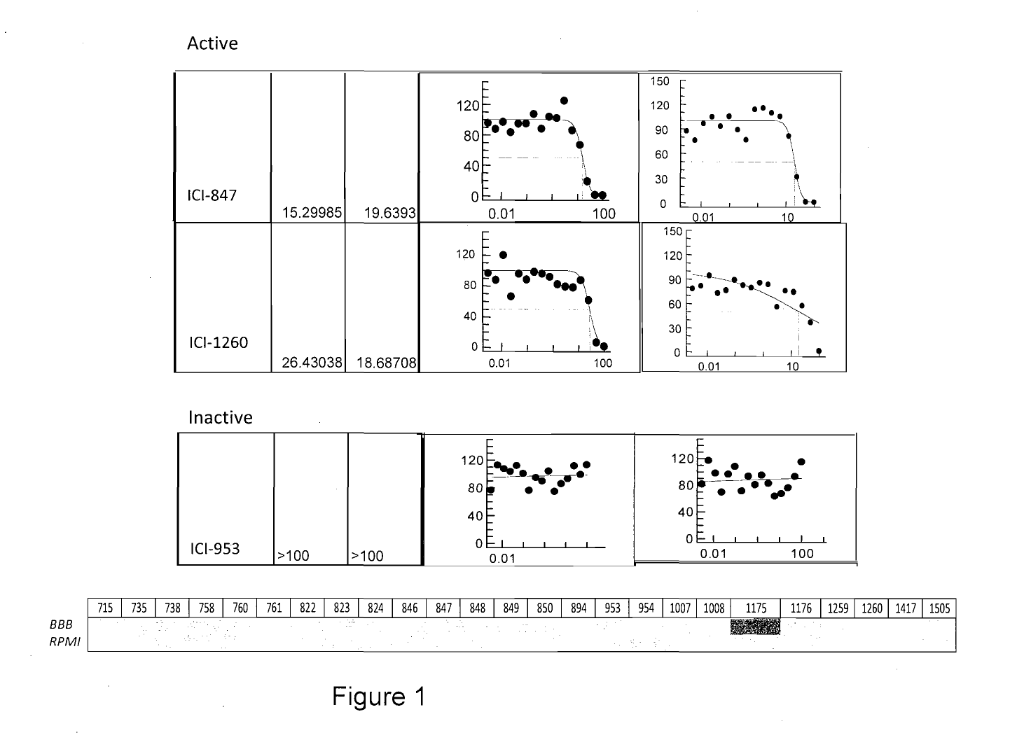

It was observed that among the compounds tested, ICI-715, ICI-761, ICI-847, ICI-848, ICI-849, ICI-1259, and ICI-1260 was able to inhibit the proliferation of RPMI cells. An exemplary depiction of compounds that are active or otherwise able to inhibit proliferation of RPMI cells and inactive compounds (e.g., ICI-953) or otherwise not able to inhibit proliferation of RPMI cells is shown in FIG. 1. FIG. 1 is a graph depicting the effects on the test compounds on RPMI cell viability.

### Example 2

### Treatment of Arthritis Using Compounds of the Invention

The next set of experiments was designed to test the ability of the compounds of the invention on a rat model of collagen-induced arthritis. Briefly, Female Lewis rats were immunized with bovine type 2 collagen 2 mg/mL in 0.01N HAc mixed 1:1 w/ FIA injected id 0.1 mL at each of three sites on back (Rosloniec, E. F., A. H. Kang, L. K. Myers, M. A. Cremer. 1996. Collagen-induced arthritis. R. Coico, and E. Shevach, eds. Current Protocols in Immunology 15.5.1. Weily & Sons, New York.).

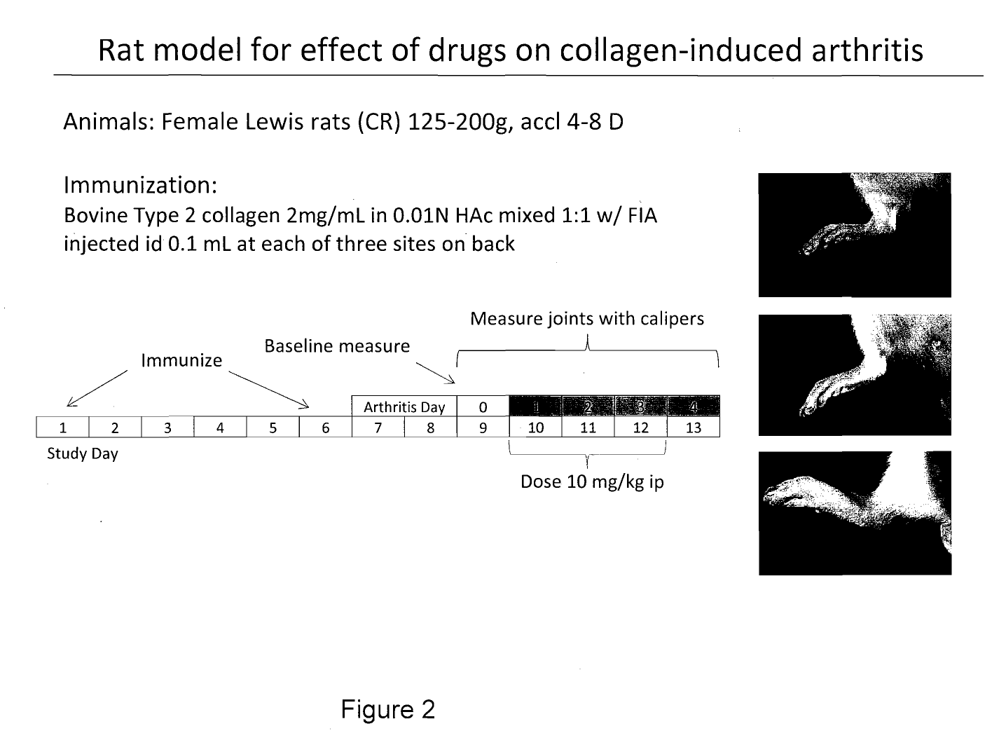

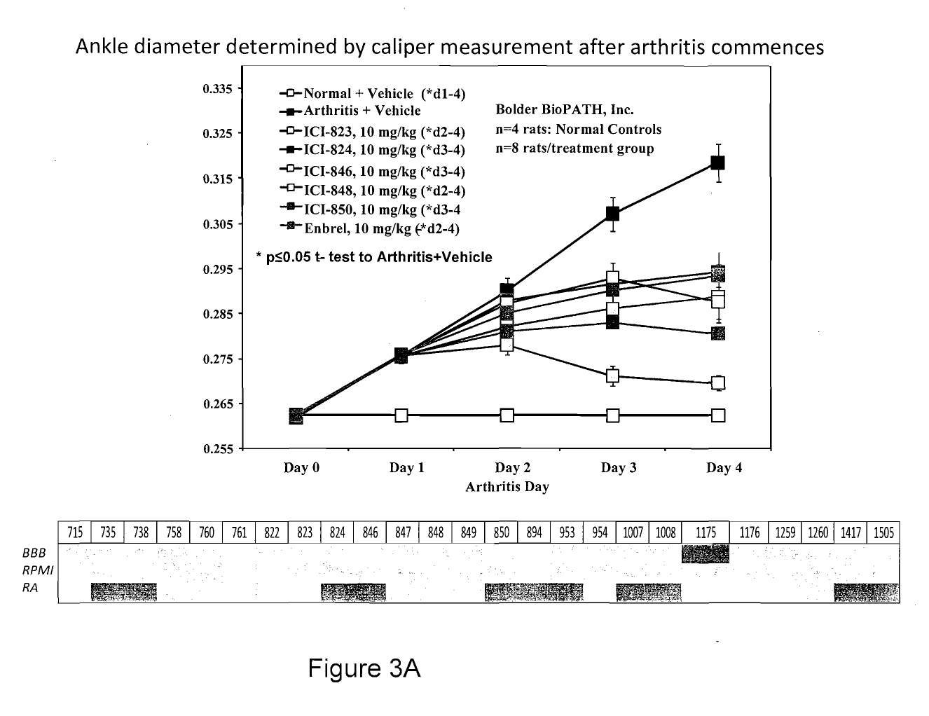

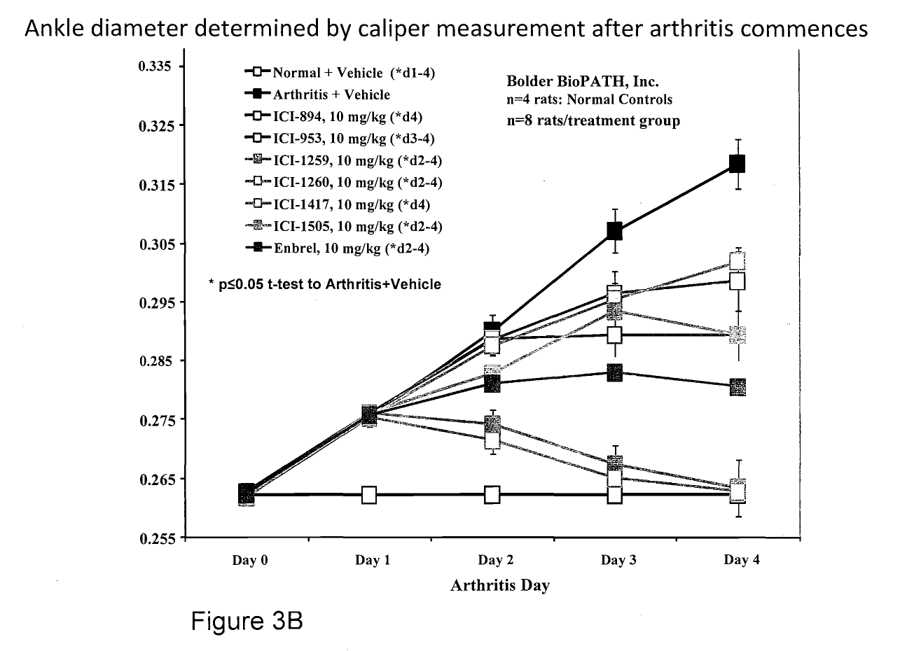

FIG. 2 depicts a schematic of the collagen-induced arthritis protocol. Briefly, a test compound of the invention is delivered ip at 10 mg/kg daily, for about 3 days. Of the compounds tested, ICI-715, 761, 823, 847, 848, 849, 1259, and 1260 were observed to decrease the ankle diameter to a desirable level as determined by caliper measurement after commencement of arthritis (FIG. 3).

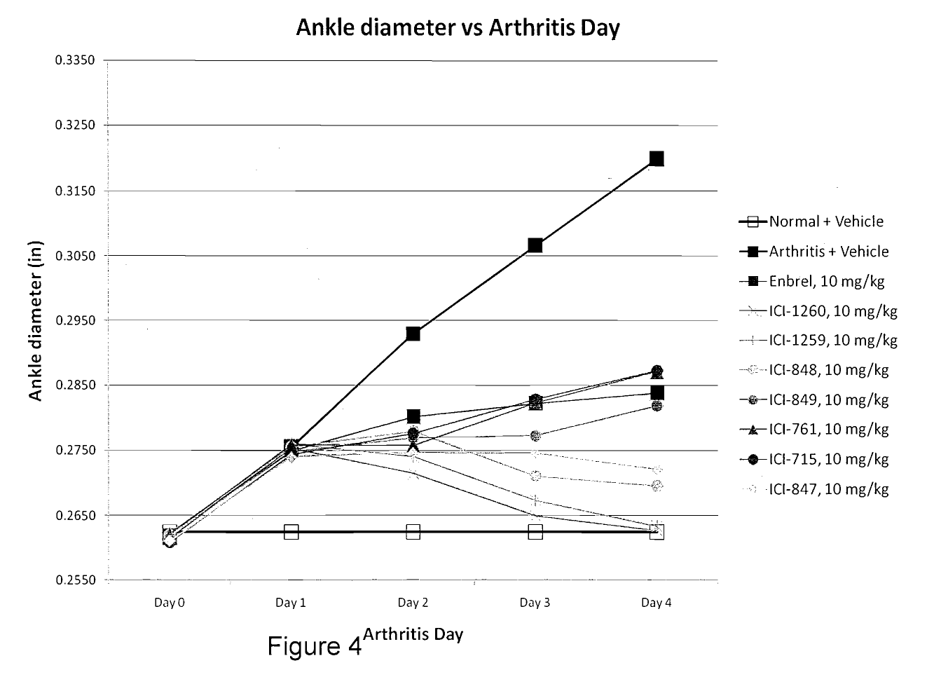

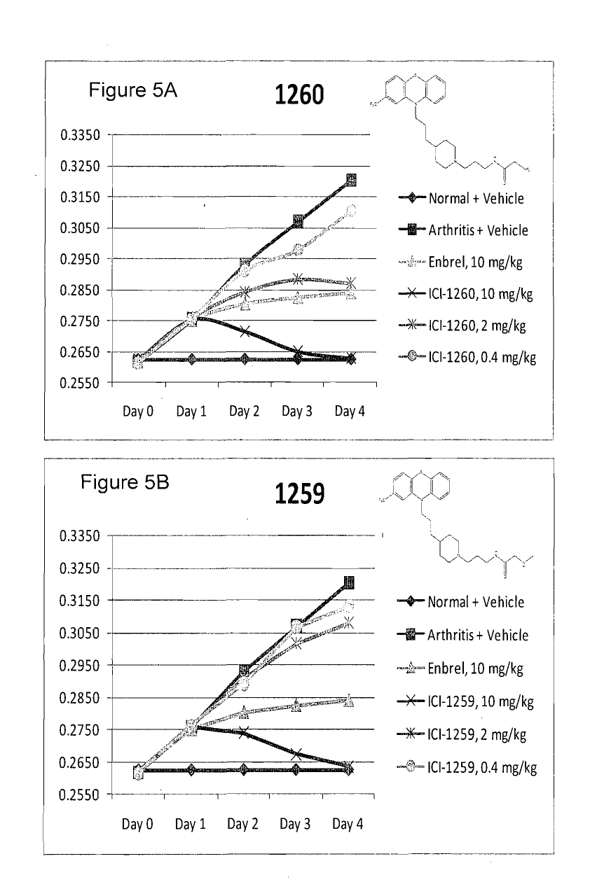

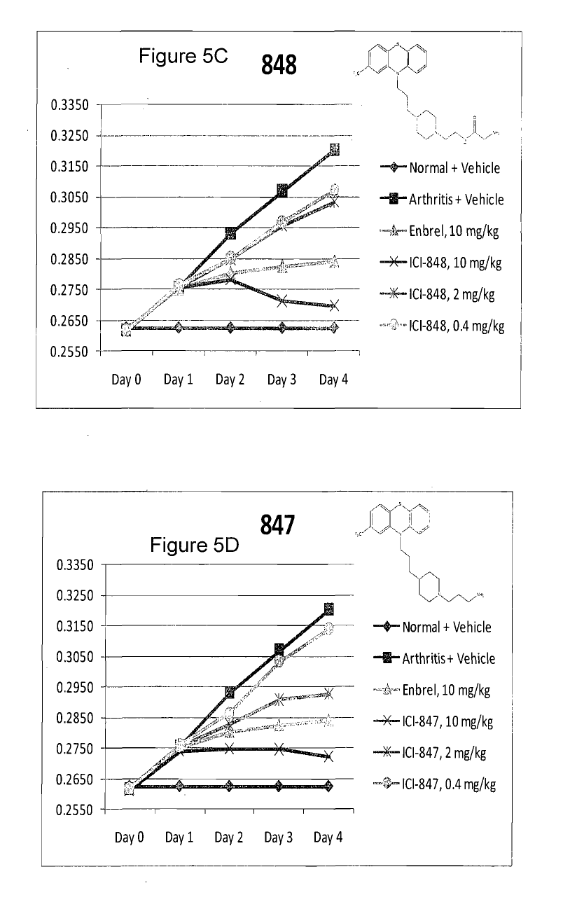

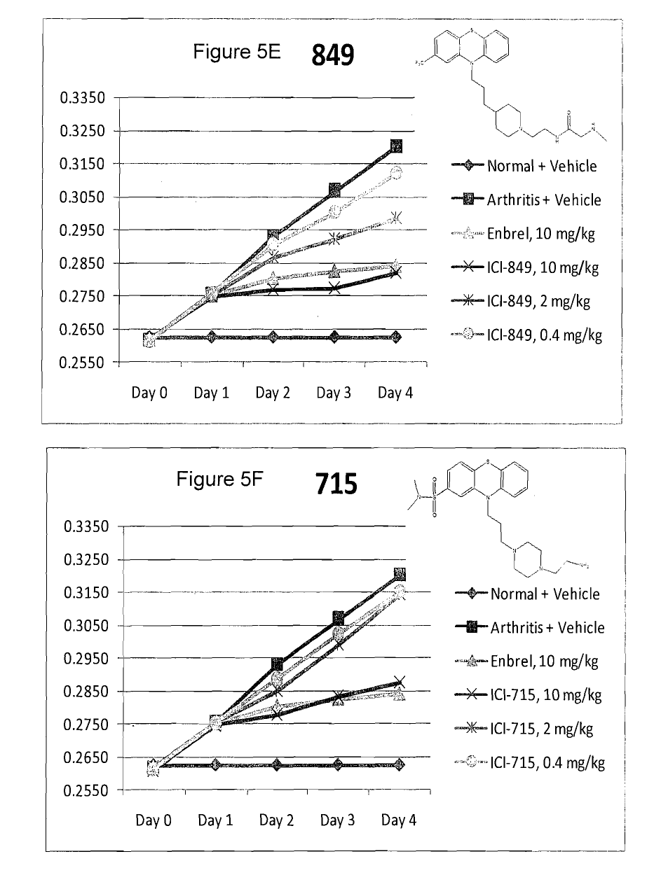

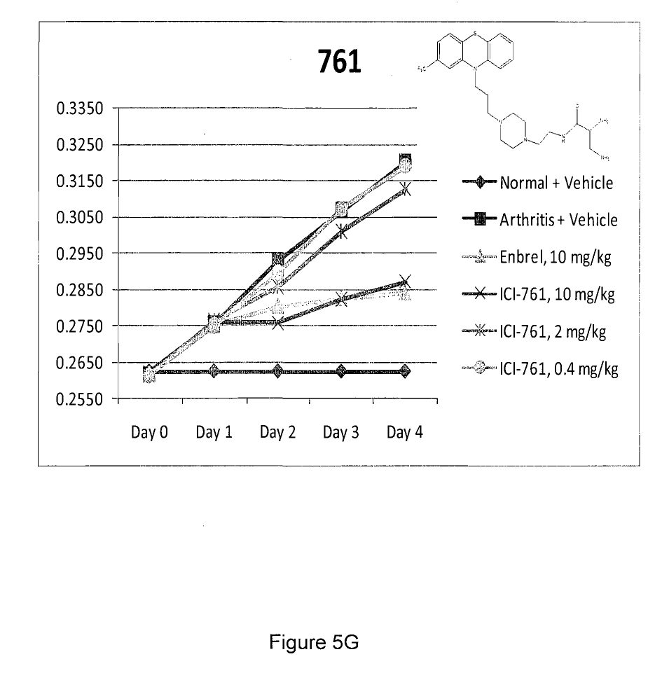

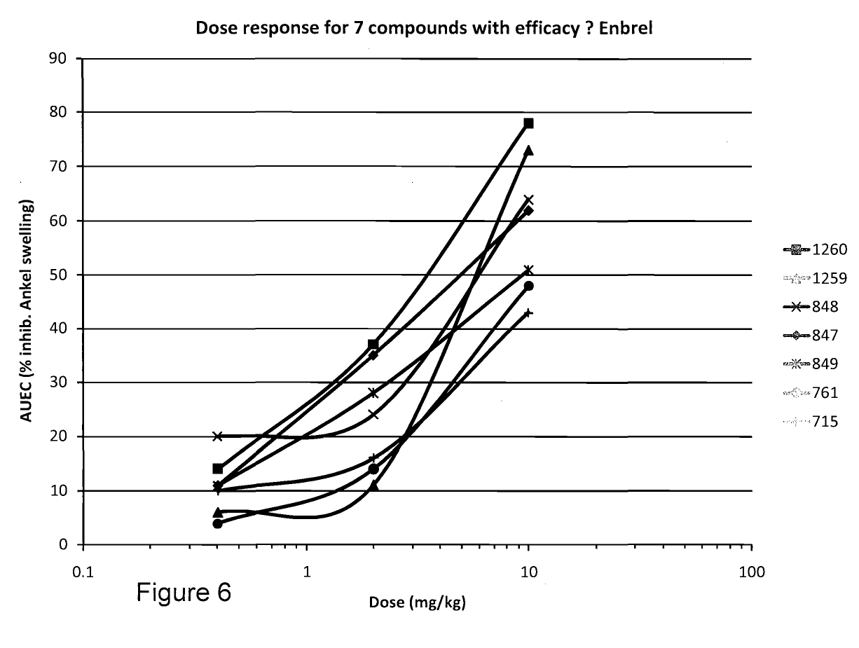

Seven of the test compounds were classified as most active compounds in the rheumatoid arthritis model. These compounds were observed to be more effective in reducing the ankle diameter than Enbrel. FIG. 4 depicts ICI 1260, 1259, 848, 849, 761, 715, and 847 with respect to Enbrel. Additional testing for the effectiveness of compounds ICI 1260, 1259, 848, 849, 761, 715, and 847 at various concentrations (10 mg/kg, 2 mg/kg, 0.4 mg/kg) were conducted, as illustrated in FIGS. 5A-5G. A dose response curve was constructed to illustrate the efficacy of the seven compounds (FIG. 6).

The next set of experiments was designed to determine the toxicity of the compounds. Each compound was administered to Female Lewis rats at a dose of 100 mg/kg via ip administration. It was observed that ICI-715, 738, 758, 823, 824, 848, 1259, and 1260 were not toxic in comparison with PBS control.

Based on the experiments to assess penetration of the blood-brain barrier, inhibition of RPMI, efficacy in a rheumatoid arthritis rat model, and toxicity, the lead compounds for further development of clinical trials include ICI-848, 849, 1259, 1260. The present experimental results therefore suggest that compounds of the invention can be useful for treating arthritis and related conditions.

### Example 3

### Serotonin-Receptor Binding Assays

The methods employed in this study have been adapted from the scientific literature to maximize reliability and reproducibility. Where presented, IC₅₀ values were determined by a non-linear, least squares regression analysis using the DATA ANALYSIS TOOLBOX (MDL Information Systems, San Leandro, Calif., USA). Where inhibition constants (Kᵢ) are presented, the Kᵢ values were calculated using the equation of Cheng and Prusoff (Cheng, Y., Prusoff, W. H., Biochem. Pharmacol. 22:3099-3108, 1973) or equivalent using the observed IC₅₀ of the tested compound, the concentration of ligand employed in the assay, and historical values for the K_{D} of the ligand.

The experiments include contacting a serotonin type 1B, 2A, 2B, 2C, 3, 4, 6, and 7 receptor with a test compound and comparing the level of binding of serotonin or a control ligand with that serotonin type 1B, 2A, 2B, 2C, 3, 4, 6, and 7 receptor with the level of serotonin binding or control ligand binding with an otherwise identical serotonin type 1B, 2A, 2B, 2C, 3, 4, 6, and 7 receptor not contacted with the test compound. The routineer would understand that a lower level of serotonin binding or control ligand binding with the receptor contacted with the compound compared with the level of serotonin binding or control ligand binding with the otherwise identical serotonin type 1B, 2A, 2B, 2C, 3, 4, 6, and 7 receptor not contacted with the compound is an indication that the compound inhibits the serotonin/receptor or control ligand/receptor interaction and is, therefore, useful for treating a disease associated with the immune system or an allogeneic graft response in a mammal. The skilled artisan would also appreciate, in view of the disclosure provided herein, that standard binding assays known in the art, or those to be developed in the future, can be used to assess the binding of serotonin or a control ligand with a serotonin type 1B. 2A, 2B, 2C, 3, 4, 6, and 7 receptor in the presence or absence of the test compound to identify a useful compound.

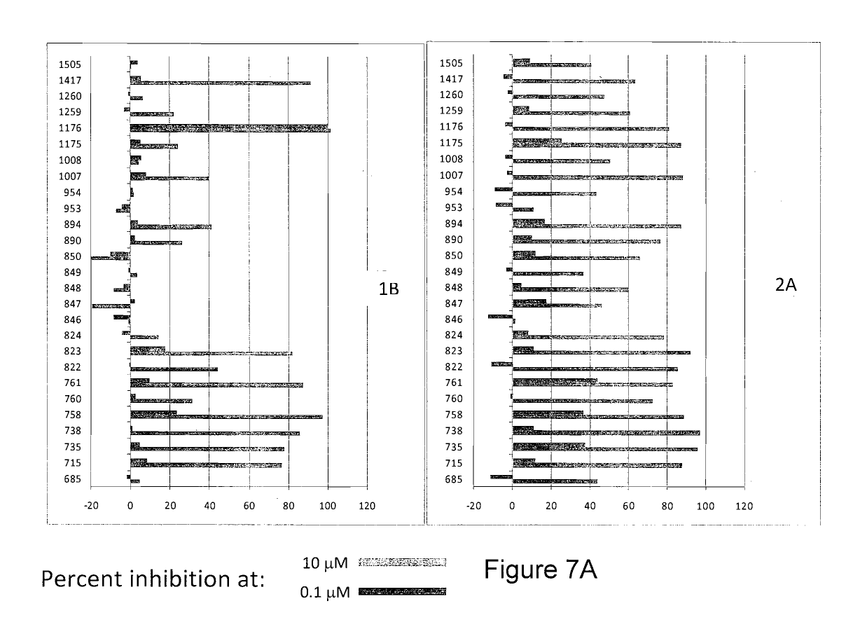

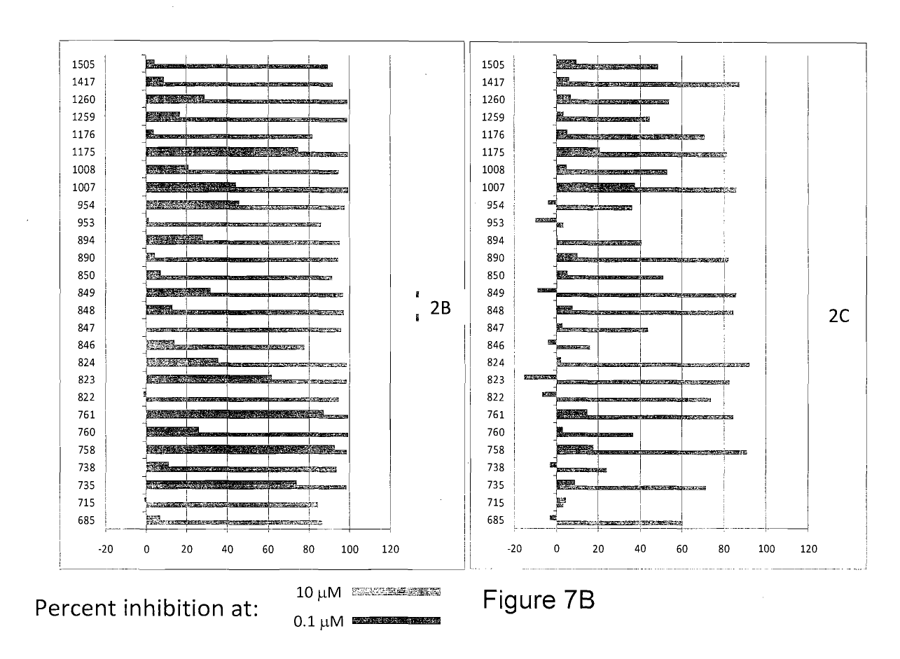

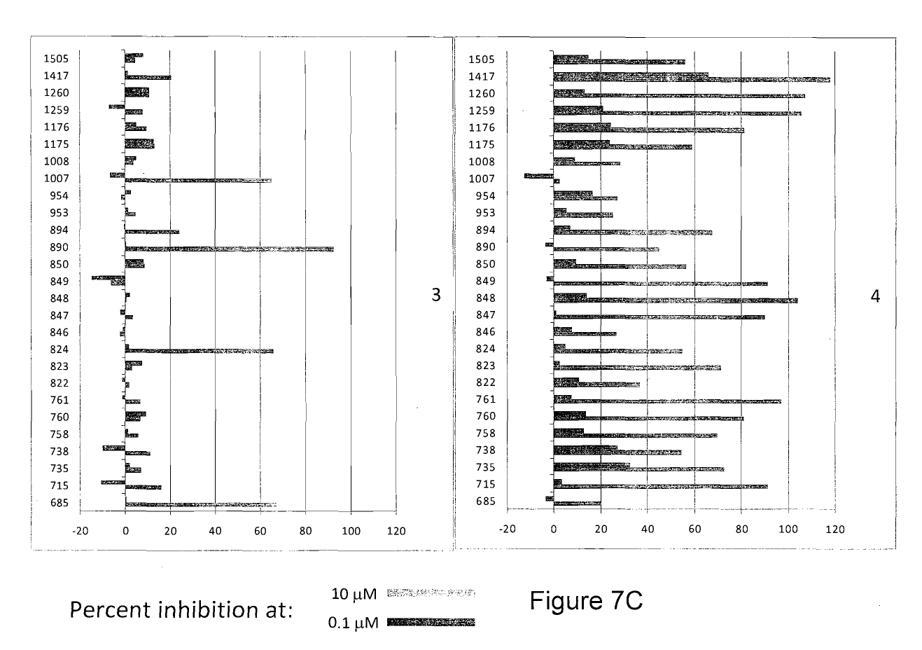

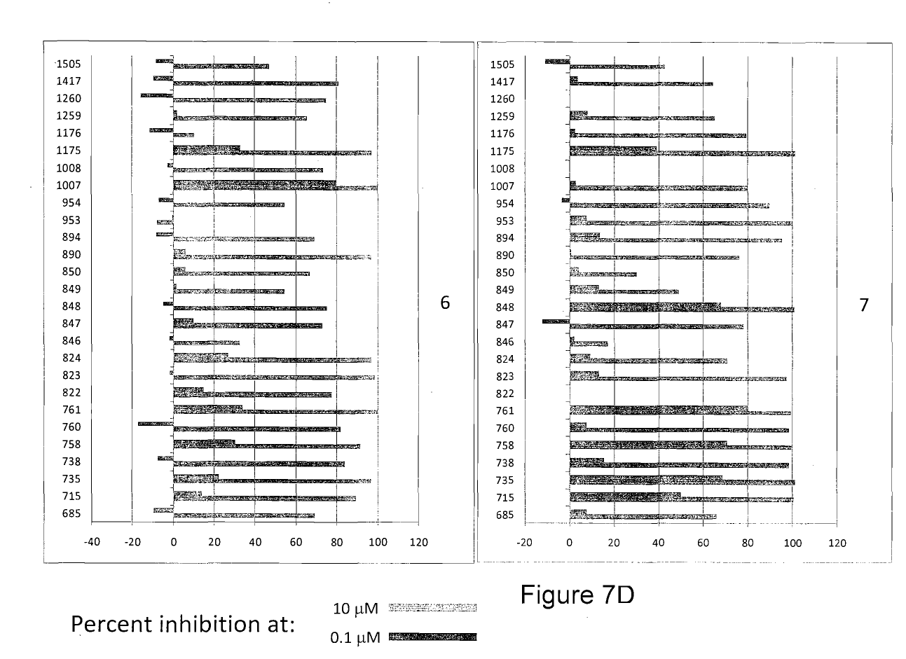

The results from the binding experiments are summarized in FIG. 7. ICI-1176 is fluphenazine. Each candidate compound was tested for selective binding to 5-HT1B, 2A, 2B, 2C, 3, 4, 6, and 7 receptors at concentrations of 10 μM and 0.1 μM.

### Example 4

### Correlation Between Percent Reduction in Ankle Diameter and 5-HT Receptor Binding

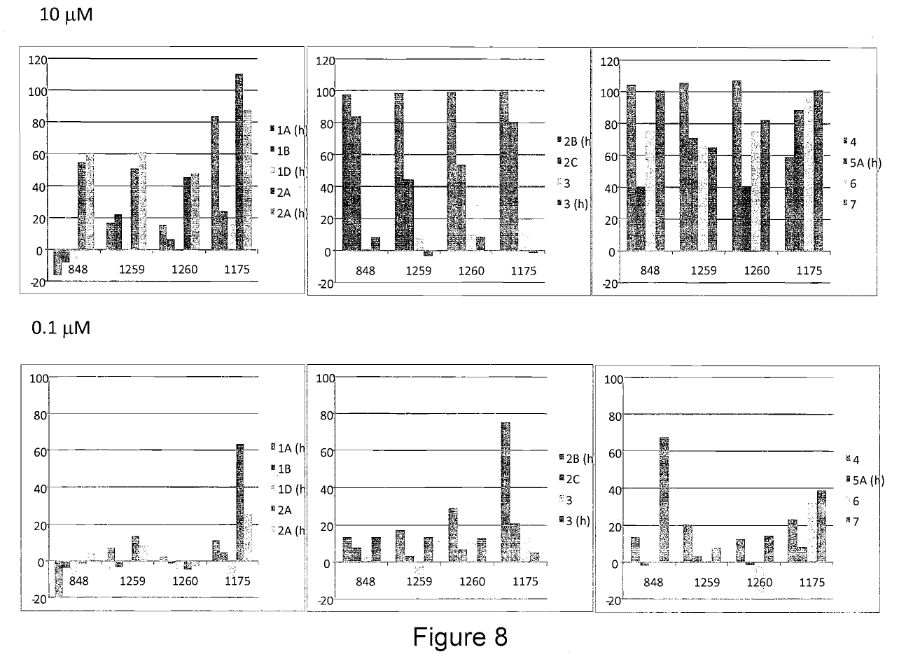

The next set of experiments was designed to assess the selective binding of the test compounds to 5-HT receptors. ICI-848, ICI-1259, and ICI-1260 were tested against fluphenazine (ICI-1175) to determine selective binding to 5-HT receptors. It was observed that the tested compounds exhibited a decreased binding to 5-HT1A and 5-HT2A receptors compared to fluphenazine. Whereas the test compounds exhibited an increased binding to 5-HT4 receptor compared to fluphenazine (FIG. 8).

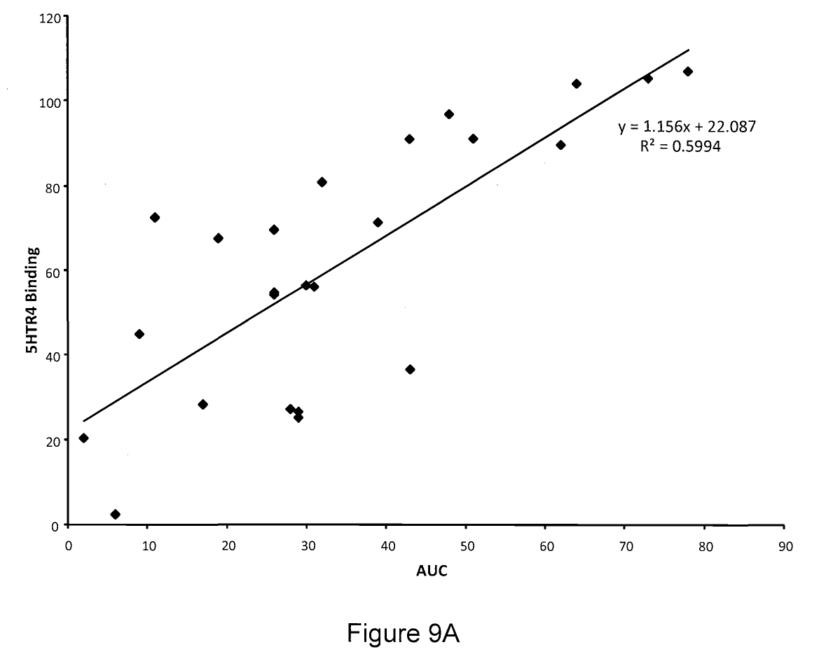

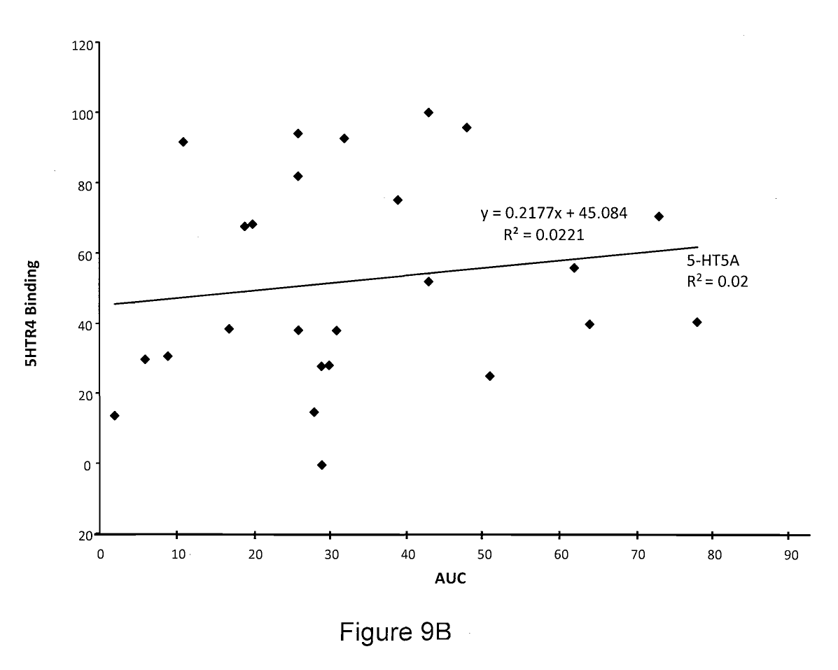

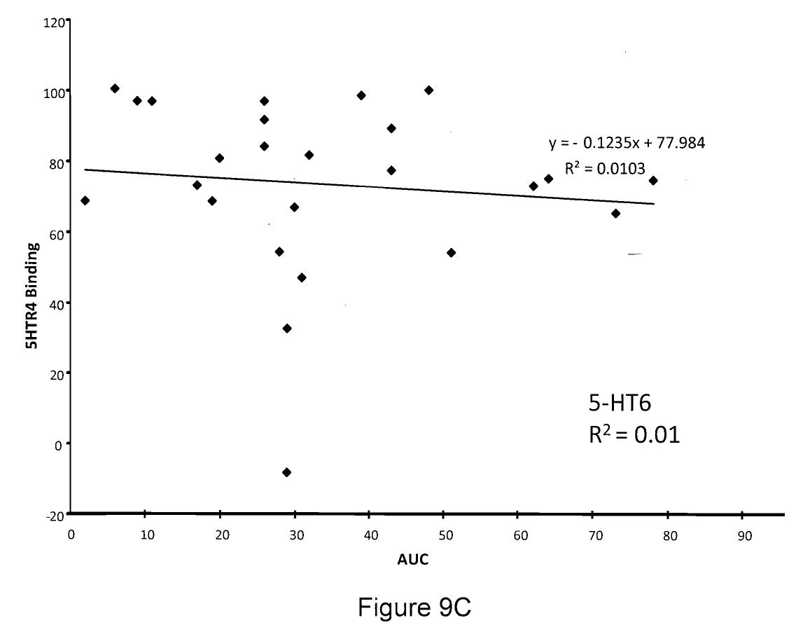

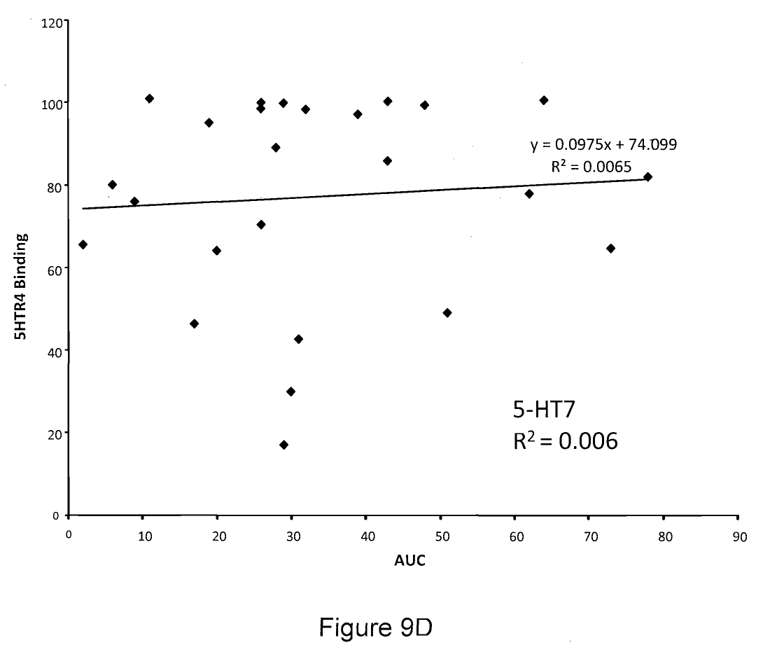

The next set of experiments was designed to compare the results from the rheumatoid arthritis rat model and 5-HT receptor binding studies. It was observed that compounds that reduced ankle diameter in the rheumatoid arthritis rat model had a greater binding affinity to 5-HT4 receptor than 5-HT5A, 5-HT6, and 5-HT7 receptors (FIG. 9).

The next set of experiments was performed to determine the dose range for which 5-HT4 receptor binding by the tested compound correlates with reduction in ankle diameter. The results demonstrate that 5-HT4 receptor binding correlates with reduced ankle diameter in a rheumatoid arthritis rat model at higher doses (e.g., 10 mg/kg) rather than lower doses (e.g., 2 mg/kg) (FIG. 9).

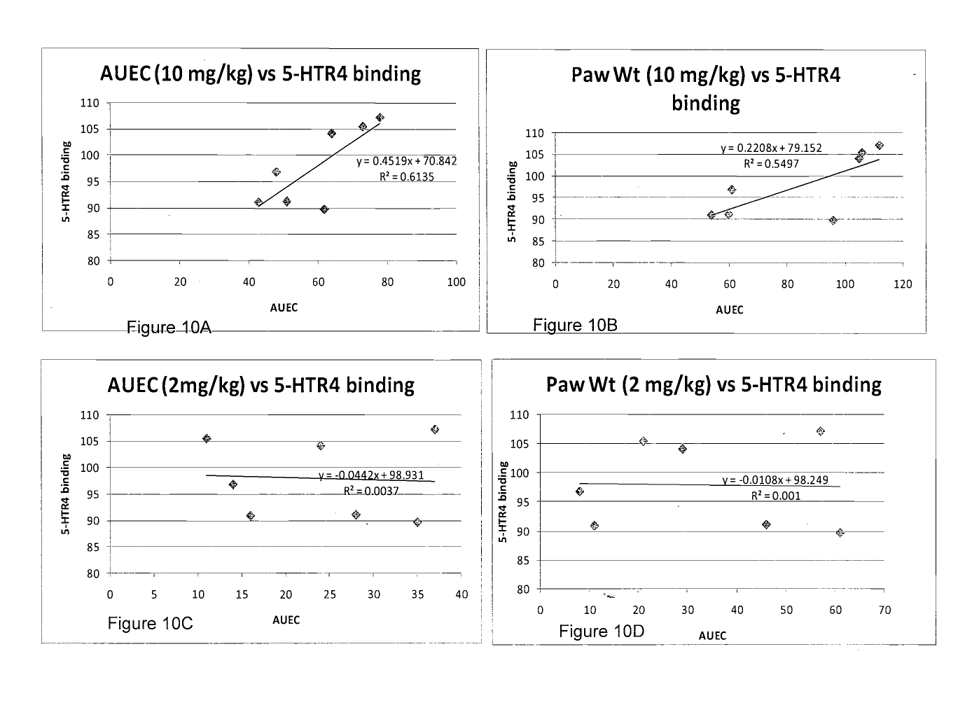

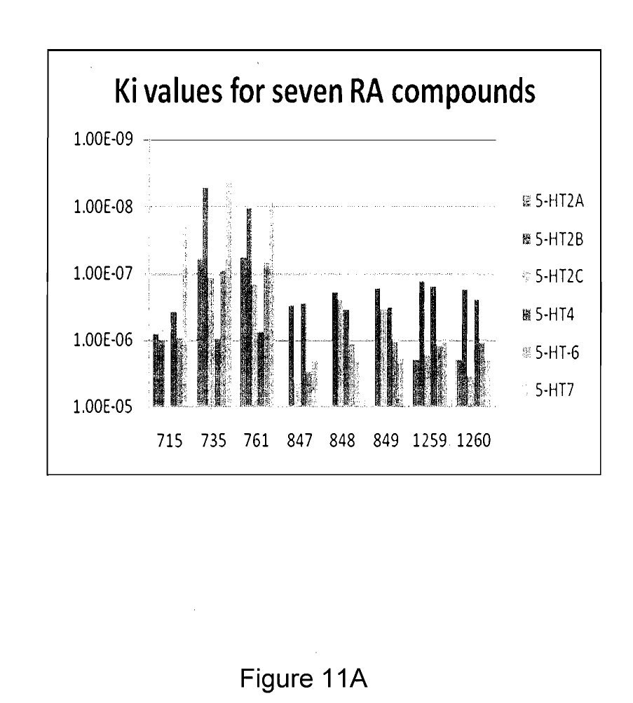

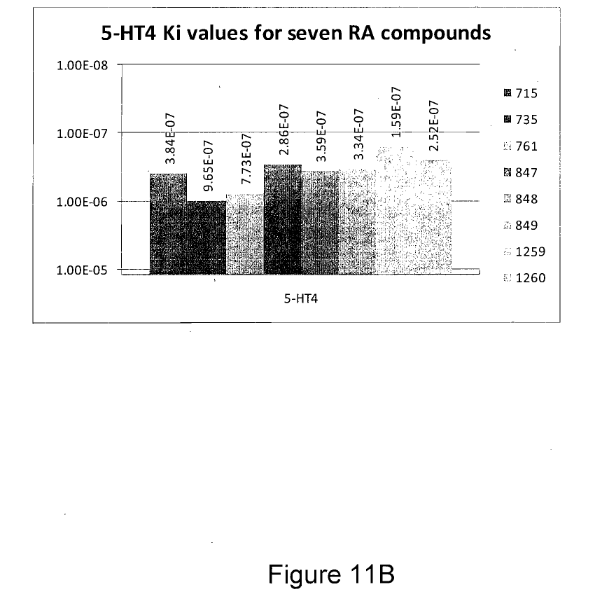

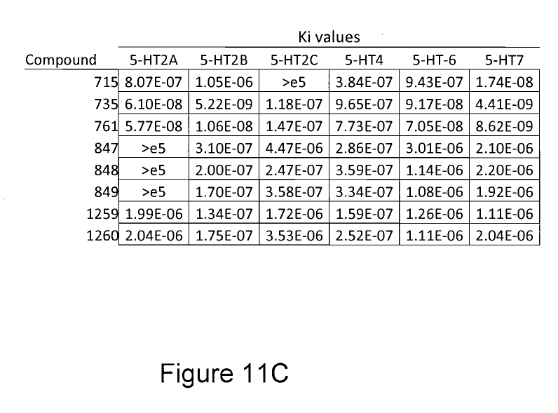

The next set of experiments was designed to assess the Ki values of the tested compounds. Of the tested compounds (ICI-715, 735, 761, 847, 848, 849, 1259, and 1260), only 735 did not have a desirable reduction of ankle diameter in a rheumatoid arthritis rat model. Moreover, 847, 848, 849, 1259, and 1260 had the most desirable reduction of ankle diameter in an arthritis rat model. ICI-847, 848, 849, 1259, and 1260 exhibited a similar Ki value profile for 5-HT2A, 5-HT2B, 5-HT2C, 5-HT4,5-HT6, and 5-HT7 receptor binding. For example, these compounds exhibited a similar Ki value with respect to 5-HT2B and 5-HT4 receptor binding. More specifically, the Ki value for 5-HT4 receptor with respect to ICI-847, 848, 849, 1259, and 1260 was within the range of 1.59⁻⁷ and 3.59⁻⁷. In addition, the Ki value for 5-HT2B receptor with respect to ICI-847, 848, 849, 1259, and 1260 was within the range of 1.34-7 and 3.10⁻⁷ (FIG. 10).

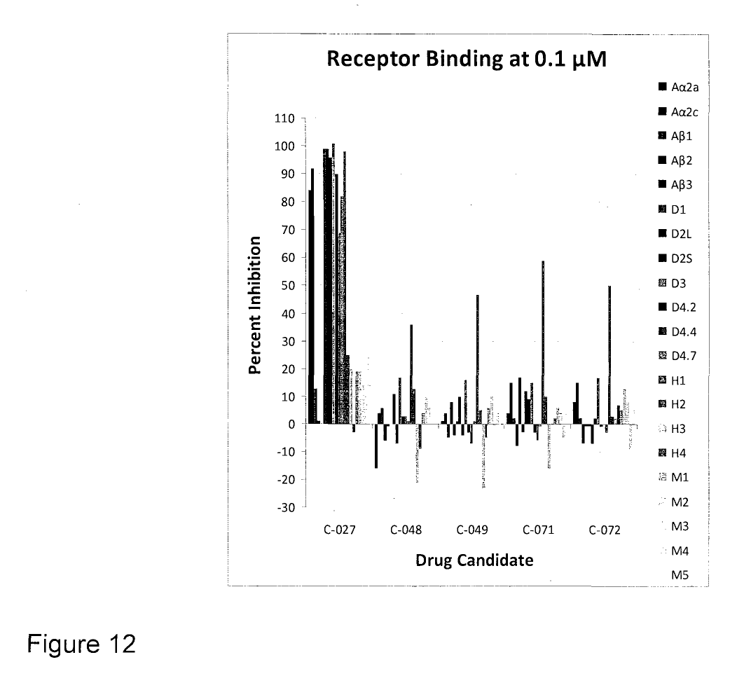

The next set of experiments was designed to assess the selective binding of the test compounds to non-5-HT receptors. The compounds tested are as follows: ICI-735, 848, 849, 1259, and 1260. It was observed that the tested compounds were able to bind histamine type 1 receptor (FIG. 12). It was also observed that the compounds that bound well to 5-HT4 receptor also bound well to histamine type 1 receptor.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising an effective amount of a 5-HT4 receptor ligand, wherein said ligand has a Ki value of less than about 1 μM for binding thereof to a 5-HT4 receptor, and further wherein said ligand is capable of modulating activity of an immune cell.

2. The composition of claim 1, wherein said ligand is a compound of formula I: 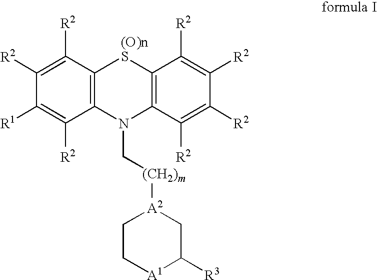
or a pharmaceutically acceptable salt, prodrug or solvate thereof, wherein:
R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A¹ is CH₂, N((CH₂)ₚNR⁷₂)₂, or NR⁴;
A² is CH or N;
provided that if A¹ is CH₂, then A² is N, and if A² is CH, then A¹ is NR⁴ or N((CH₂)ₚNR⁷₂)₂
R⁴ is H, (C₁-C₆)alkyl; heteroaryl; (CH₂)ₚOR⁷; (CH₂)ₚNR⁷₂; (CH₂)ₚNHC(O)R⁵; (CH₂)ₚO(CH₂)ₚOR⁷; (CH₂)ₚO(CH₂)ₚNR⁷₂; (CH₂)ₚNR⁴(CH₂)ₚNR⁷₂; (CH₂)ₚO(CH₂)ₚNHC(O)R⁵; (CH₂)ₚNR⁷(CH₂)ₚNHC(O)R⁵; (CH₂)_{q}C(═O)OR⁷; (CH₂)_{q}C(═O)NR⁷₂; (CH₂)ₚO(CH₂)_{q}C(═O)OR⁷; (CH₂)ₚO(CH₂)_{q}C(═O)NR⁷₂; (CH₂)ₚNR⁷(CH₂)_{q}C(═O)OR⁷; (CH₂)ₚNR⁷(CH₂)_{q}C(═O)NR⁷₂; (CH₂)ₚR⁸; C(═O)(CH₂)ₚR⁸; (CH₂)ₚO(CH₂)ₚNR⁸, (CH₂)ₚNR⁴(CH₂)ₚNR⁸; (CH₂)_{q}C(═O)NR⁸, (CH₂)ₚO(CH₂)_{q}C(═O)NR⁸, (CH₂)ₚNR⁷(CH₂)_{q}C(═O)NR⁸ or C(═O)(CH₂)ₚNR⁷₂;
R⁵ is (C₁-C₆)alkyl; NR⁷C(═O)(C₁-C₆)alkyl; NR⁷C(═O)O(C₁-C₆)alkyl; NR⁷C(═O)NR⁷₂; CH(R⁶)NR⁷₂; CH(R⁶)NR⁷C(═O)(C₁-C₆)alkyl; (1H-pyrrolidin-2-yl), or CH(R⁶)NR⁷C(═O)O(C₁-C₆)alkyl.
R⁶ is H, (C₁-C₆)alkyl; (C₁-C₆)alkylene-OR⁷; (C₁-C₆)alkylene-NH—C(═NH)—NH₂; (C₁-C₆)alkylene-NR⁷₂; (C₁-C₆)alkylene-SR⁷; benzyl; 4′-hydroxybenzyl; (CH₂)_{q}C(═O)OR⁷; or (CH₂)_{q}C(═O)NR⁷₂;
R⁷ is independently selected at each occurrence from the group consisting of hydrogen and (C₁-C₆)alkyl;
R⁸ is 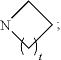
m is independently at each occurrence 1, 2, or 3;
n is 0, 1, or 2;
p is independently at each occurrence 2 or 3;
q is independently at each occurrence 1 or 2; and
t is 1, 2 or 3.

3. The composition of claim 1, wherein said ligand is a compound of formula I: 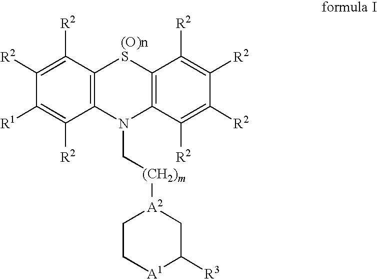
or a pharmaceutically acceptable salt, prodrug or solvate thereof, wherein:
R¹ is CF₃;
R² is hydrogen;
R³ is hydrogen;
A¹ is NR⁴;
A² is CH or N;
R⁴ is (CH₂)ₚNHC(O)R⁵;
R⁵ is (C₁-C₆)alkyl; or CH(R⁶)NR⁷₂;
R⁶ is H, or (C₁-C₆)alkyl;
R⁷ is independently selected at each occurrence from the group consisting of hydrogen and (C₁-C₆)alkyl;
m is 2;
n is 0; and
p is 2 or 3.

4. The composition of claim 1, wherein said ligand is a compound of formula I: 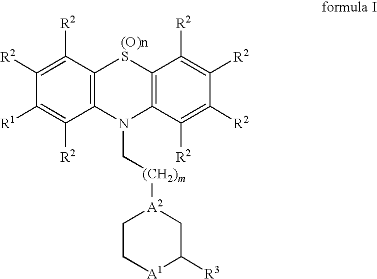
or a pharmaceutically acceptable salt, prodrug or solvate thereof, wherein:
R¹ is CF₃;
R² is hydrogen;
R³ is hydrogen;
A¹ is NR⁴;
A² is CH;
R⁴ is (CH₂)ₚNHC(O)R⁵;
R⁵ is CH₂NHR⁷;
R⁷ is hydrogen or methyl;
m is 2;
n is 0; and
p is 2 or 3.

5. The composition of claim 1, wherein said ligand inhibits proliferation of an immune cell when said ligand binds to a 5-HT4 receptor on said immune cell.

6. The composition of claim 5, wherein said immune cell is selected from the group consisting of a T cell, a B cell, a natural killer cell, a dendritic cell, and a macrophage, a monocyte, a neutrophil, a eosinophil, and a basophile.

7. The composition of claim 1, wherein said ligand does not substantially modulate cells of the central nervous system.

8. The composition of claim 1, wherein said ligand does not substantially cross the blood-brain barrier.

9. A method of treating a disease characterized by abnormal immune cell proliferation, the method comprising administering to a mammal in need thereof a composition of claim 1.

10. The method of claim 9, wherein the mammal is a human.

11. The method of claim 9, wherein the immune cell is selected from the group consisting of a T cell, a B cell, a natural killer cell, a dendritic cell, a macrophage, a monocyte, a neutrophil, a eosinophil, and a basophile.

12. The method of claim 9, wherein the administered composition does not substantially modulate central nervous system function of the mammal.

13. The method of claim 9, wherein said ligand does not substantially cross the blood-brain barrier of the mammal.

14. A method of treating rheumatoid arthritis in a mammal, said method comprising administering to a mammal in need thereof an effective amount of a composition comprising a 5-HT4 receptor ligand, wherein said ligand has a Ki value of less than about 1 μM for binding thereof to said 5-HT4 receptor, and further wherein said ligand is capable of modulating activity of an immune cell.

15. The method of claim 14, wherein said ligand is a compound of formula I: 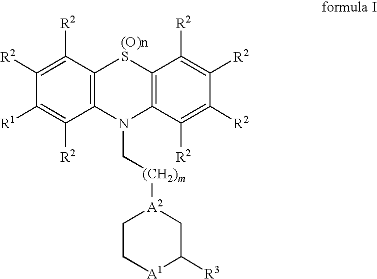
or a pharmaceutically acceptable salt, prodrug or solvate thereof, wherein:
R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A¹ is CH₂, N((CH₂)ₚNR⁷₂)₂, or NR⁷₂;
A² is CH or N;
provided that if A¹ is CH₂, then A² is N, and if A² is CH, then A¹ is NR⁴ or N((CH₂)ₚNR⁷₂)₂
R⁴ is H, (C₁-C₆)alkyl; heteroaryl; (CH₂)ₚOR⁷; (CH₂)ₚNR⁷₂; (CH₂)ₚNHC(O)R⁵; (CH₂)ₚO(CH₂)ₚOR⁷; (CH₂)ₚO(CH₂)ₚNR⁷₂; (CH₂)ₚNR⁴(CH₂)ₚNR⁷₂; (CH₂)ₚO(CH₂)ₚNHC(O)R⁵; (CH₂)ₚNR⁷(CH₂)ₚNHC(O)R⁵; (CH₂)_{q}C(═O)OR⁷; (CH₂)_{q}C(═O)NR⁷₂; (CH₂)ₚO(CH₂)_{q}C(═O)OR⁷; (CH₂)ₚO(CH₂)_{q}C(═O)NR⁷₂; (CH₂)ₚNR⁷(CH₂)_{q}C(═O)OR⁷; (CH₂)ₚNR⁷(CH₂)_{q}C(═O)NR⁷₂; (CH₂)ₚR⁸; C(═O)(CH₂)ₚR⁸; (CH₂)ₚO(CH₂)ₚNR⁸, (CH₂)ₚNR⁸,(CH₂)ₚNR⁸; (CH₂)_{q}C(═O)NR⁸, (CH₂)ₚO(CH₂)_{q}C(═O)NR⁸, (CH₂)ₚNR⁷(CH₂)_{q}C(═O)NR⁸ or C(═O)(CH₂)ₚNR⁷₂;
R⁵ is (C₁-C₆)alkyl; NR⁷C(═O)(C₁-C₆)alkyl; NR⁷C(═O)O(C₁-C₆)alkyl; NR⁷C(═O)NR⁷₂; CH(R⁶)NR⁷₂; CH(R⁶)NR⁷C(═O)(C₁-C₆)alkyl; (1H-pyrrolidin-2-yl), or CH(R⁶)NR⁷C(═O)O(C₁-C₆)alkyl.
R⁶ is H, (C₁-C₆)alkyl; (C₁-C₆)alkylene-OR⁷; (C₁-C₆)alkylene-NH—C(═NH)—NH₂; (C₁-C₆)alkylene-NR⁷₂; (C₁-C₆)alkylene-SR⁷; benzyl; 4′-hydroxybenzyl; (CH₂)_{q}C(═O)OR⁷; or (CH₂)_{q}C(═O)NR⁷₂;
R⁷ is independently selected at each occurrence from the group consisting of hydrogen and (C₁-C₆)alkyl;
R⁸ is 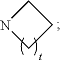
m is independently at each occurrence 1, 2, or 3;
n is 0, 1, or 2;
p is independently at each occurrence 2 or 3;
q is independently at each occurrence 1 or 2; and
t is 1, 2 or 3.

16. The method of claim 14, wherein said ligand inhibits proliferation of an immune cell associated said rheumatoid arthritis when said ligand binds to a 5-HT4 receptor on said immune cell.

17. The method of claim 16, wherein said immune cell is selected from the group consisting of a T cell, a B cell, a natural killer cell, a dendritic cell, and a macrophage, a monocyte, a neutrophil, a eosinophil, and a basophile.

18. The method of claim 14, wherein the administered composition does not substantially modulate central nervous system function of the mammal.

19. The method of claim 14, wherein said ligand does not substantially cross the blood-brain barrier of the mammal.

20. The method of claim 14, wherein said mammal is a human.

21. A method of treating rheumatoid arthritis in a mammal, said method comprising administering to a mammal in need thereof an effective amount of a composition comprising a 5-HT4 receptor ligand in combination with a therapeutic agent, wherein said therapeutic agent is a rheumatoid arthritis medicament and wherein said ligand has a Ki value of less than about 1 μM for binding thereof to said 5-HT4 receptor, and further wherein said ligand is capable of modulating activity of an immune cell.

22. The method of claim 21, wherein said ligand is a compound of formula I: 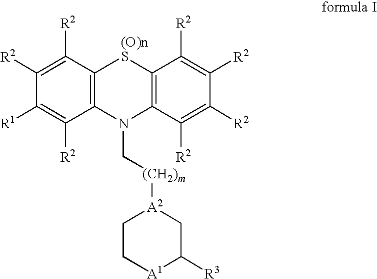
or a pharmaceutically acceptable salt, prodrug or solvate thereof, wherein:
R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A¹ is CH₂, N((CH₂)ₚNR⁷₂)₂, or NR⁴;
A² is CH or N;
provided that if A¹ is CH₂, then A² is N, and if A² is CH, then A¹ is NR⁴ or N((CH₂)ₚNR⁷₂)₂
R⁴ is H, (C₁-C₆)alkyl; heteroaryl; (CH₂)ₚOR⁷; (CH₂)ₚNR⁷₂; (CH₂)ₚNHC(O)R⁵; (CH₂)ₚO(CH₂)ₚOR⁷; (CH₂)ₚO(CH₂)ₚNR⁷₂; (CH₂)ₚNR⁴(CH₂)ₚNR⁷₂; (CH₂)ₚO(CH₂)ₚNHC(O)R⁵; (CH₂)ₚNR⁷(CH₂)ₚNHC(O)R⁵; (CH₂)_{q}C(═O)OR⁷; (CH₂)_{q}C(═O)NR⁷₂; (CH₂)ₚO(CH₂)_{q}C(═O)OR⁷; (CH₂)ₚO(CH₂)_{q}C(═O)NR⁷₂; (CH₂)ₚNR⁷(CH₂)_{q}C(═O)OR⁷; (CH₂)ₚNR⁷(CH₂)_{q}C(═O)NR⁷₂; (CH₂)ₚR⁸; C(═O)(CH₂)ₚR⁸; (CH₂)ₚO(CH₂)ₚNR⁸, (CH₂)ₚNR⁴(CH₂)ₚNR⁸; (CH₂)_{q}C(═O)NR⁸, (CH₂)ₚO(CH₂)_{q}C(═O)NR⁸, (CH₂)ₚNR⁷(CH₂)_{q}C(═O)NR⁸ or C(═O)(CH₂)ₚNR⁷₂;
R⁵ is (C₁-C₆)alkyl; NR⁷C(═O)(C₁-C₆)alkyl; NR⁷C(═O)O(C₁-C₆)alkyl; NR⁷C(═O)NR⁷₂; CH(R⁶)NR⁷₂; CH(R⁶)NR⁷C(═O)(C₁-C₆)alkyl; (1H-pyrrolidin-2-yl), or CH(R⁶)NR⁷C(═O)O(C₁-C₆)alkyl.
R⁶ is H, (C₁-C₆)alkyl; (C₁-C₆)alkylene-OR⁷; (C₁-C₆)alkylene-NH—C(═NH)—NH₂; (C₁-C₆)alkylene-NR⁷₂; (C₁-C₆)alkylene-SR⁷; benzyl; 4′-hydroxybenzyl; (CH₂)_{q}C(═O)OR⁷; or (CH₂)_{q}C(═O)NR⁷₂;
R⁷ is independently selected at each occurrence from the group consisting of hydrogen and (C₁-C₆)alkyl;
R⁸ is 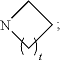
m is independently at each occurrence 1, 2, or 3;
n is 0, 1, or 2;
p is independently at each occurrence 2 or 3;
q is independently at each occurrence 1 or 2; and
t is 1, 2 or 3.

23. The method of claim 21, wherein said ligand inhibits proliferation of an immune cell associated said rheumatoid arthritis when said ligand binds to a 5-HT4 receptor on said immune cell.

24. The method of claim 23, wherein said immune cell is selected from the group consisting of a T cell, a B cell, a natural killer cell, a dendritic cell, and a macrophage, a monocyte, a neutrophil, a eosinophil, and a basophile.

25. The method of claim 21, wherein the administered composition does not substantially modulate central nervous system function of the mammal.

26. The method of claim 21, wherein said ligand does not substantially cross the blood-brain barrier of the mammal.

27. The method of claim 21, wherein said mammal is a human.

28. The method of claim 21, wherein said therapeutic agent is administered simultaneously, prior to, or after administration of said composition.
